(19) United States
(12) Patent Application Publication
(10) Pub. No.: US 2003/0198980 A1
(43) Pub. Date: Oct. 23, 2003

(54) Heteroconfigurational polynucleotides and methods of use

(75) Inventors: I. Lawrence Greenfield, San Mateo, CA (US); Stefan M. Matysiak, Montara, CA (US); Benjamin Schroeder, San Mateo, CA (US); Ravi Vinayak, Mountain View, CA (US)

Correspondence Address: MILA KASAN, PATENT DEPT., APPLIED BIOSYSTEMS, 850 LINCOLN CENTRE DRIVE, FOSTER CITY, CA, 94404, US

(73) Assignee: Applera Corporation, Foster City, CA

(21) Appl. No.: 10/328,307

(22) Filed: Dec. 23, 2002

Related U.S. Application Data
(60) Provisional application No. 60/343,519, filed on Dec. 21, 2001.

(51) Int. Cl. C12Q 1/68; C07H 21/02; C12M 1/34; C07K 14/00

(52) U.S. Cl. 435/6; 435/287.2; 530/350; 536/23.1

(57) ABSTRACT

Methods, compositions and kits are disclosed that utilize heteroconfigurational polynucleotide comprising a D-form polynucleotide sequence portion and an L-form polynucleotide sequence portion that is covalently linked to the D-form polynucleotide sequence portion.

### FIELD OF THE INVENTION

The invention relates to methods and compositions for detection of nucleic acids using L-DNA.

### INTRODUCTION

Nucleic acid detection assays are important tools in molecular biology research and for medical diagnostics. Numerous nucleic acid probe assays that detect specific nucleic acid sequences in samples are based on the detection of signals that indicate hybridization, ligation, primer extension, and copying events. Nucleic acid detection is key in assays that identify microorganisms, monitor gene expression, and type and identify tissue and blood samples.

A variety of DNA hybridization techniques are available for detecting the presence of one or more selected polynucleotide sequences in a sample containing a large number of sequence regions. In a simple method, which relies on fragment capture and labeling, a nucleic acid fragment containing a selected sequence is captured by hybridization to an immobilized probe. The captured fragment can be labeled by hybridization to a second probe which contains a detectable reporter moiety. Alternatively, the nucleic acid fragment can be labelled prior to capture, by a variety of procedures including primer-extension incorporation of labelled nucleotides, amplification with labelled primers, chemical labelling reactions, ligation of labelled probes, and cross-linking of hybridization complexes.

One shortcoming of existing assays is that cross-hybridization between probes and unintended target sequences or between different probes can interfere with assay performance. Accordingly, improvements are needed avoid such cross-hybridization while maintaining good assay performance.

### SUMMARY OF THE INVENTION

In one aspect, the invention includes a polynucleotide composition comprising a heteroconfigurational polynucleotide comprising a D-form polynucleotide sequence portion and an L-form polynucleotide sequence portion that is covalently linked to the D-form polynucleotide sequence portion. In some embodiments, the L-form polynucleotide sequence portion comprises 5 to 50 L-nucleotides. In some embodiments, the D-form polynucleotide sequence portion comprises 5 to 50 D-nucleotides.

In some embodiments, the L-form polynucleotide sequence portion comprises at least one L-form 2′-4′ LNA nucleotide. In some embodiments, the L-form polynucleotide sequence portion comprises at least one L-form nucleotide comprising a 1′-α-anomeric nucleotide or a 4′-α-anomeric nucleotide. In some embodiments, the L-form polynucleotide sequence portion comprises at least one L-form nucleotide comprising ribose, arabinose, xylose, or pyranose, in the 1′-β anomeric configuration. In some embodiments, the L-form polynucleotide sequence portion comprises at least one L-form nucleotide comprising ribose, arabinose, xylose, or pyranose, in the 1′-α anomeric configuration. In some embodiments, the L-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose. In some embodiments, the D-form polynucleotide sequence portion comprises at least one D-form 2′-4′ LNA nucleotide. In some embodiments, the D-form polynucleotide sequence portion comprises at least one L-form nucleotide comprising a 1′-α-anomeric nucleotide or a 4′-α-anomeric nucleotide. In some embodiments, the D-form polynucleotide sequence portion comprises at least one L-form nucleotide comprising ribose, arabinose, xylose, or pyranose, in the 1′-β anomeric configuration. In some embodiments, the D-form polynucleotide sequence portion comprises at least one L-form nucleotide comprising ribose, arabinose, xylose, or pyranose, in the 1′-α anomeric configuration. In some embodiments, the D-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose. In some embodiments, at least one of the D-form polynucleotide sequence portion and the L-form polynucleotide sequence portion comprises an internucleotide linkage selected from a 2-aminoethylglycine, a phosphorothioate, a phosphorodithioate, a phosphotriester, and a phosphoramidate.

In some embodiments, the composition of any one of the preceding claims, wherein the heteroconfigurational polynucleotide comprises a nucleobase selected from uracil, thymine, cytosine, adenine, 7-deazaadenine, guanine, and 7-deazaguanosine.

In some embodiments, the heteroconfigurational polynucleotide comprises a nucleobase selected from 2,6-diaminopurine, hypoxanthine, pseudouridine, C-5-propyne, isocytosine, isoguanine, and 2-thiopyrimidine.

In some embodiments, the composition comprises a first complementary polynucleotide that is hybridized to the L-form polynucleotide sequence portion. In some embodiments, the first complementary polynucleotide comprises at least one L-form nucleotide. In some embodiments, the first complementary polynucleotide comprises at least one L-form 2′ deoxyribose or 2′-4′ LNA nucleotide. In some embodiments, the first complementary polynucleotide comprises at least two peptide nucleic acid subunits.

In some embodiments, the first complementary polynucleotide is attached to a solid support. In some embodiments, the solid support comprises polystyrene, glass, silica gel, silica, polyacrylamide, polyacrylate, hydroxyethylmethacrylate, polyamide, polyethylene, polyethyleneoxy, or nylon. In some embodiments, the solid support comprises a small particle, a bead, a membrane, a frit, a slide, a plate, a micromachined chip, an alkanethiol-gold layer, a non-porous surface, an addressable array, or a gel. In some embodiments, the solid support comprises a bead, a polystyrene bead, and/or a nylon membrane. In some embodiments, the solid support comprises a small particle selected from a nanoparticle, a microsphere, or a liposome. In some embodiments, the solid support comprises glass. In some embodiments, the first complementary polynucleotide is attached to the support via a cleavable linker. In some embodiments, the cleavable linker comprises a carbonyl group through which the first complementary polynucleotide is linked to the support.

In some embodiments, the composition comprises a second complementary polynucleotide that is hybridized to the D-form polynucleotide sequence portion.

In some embodiments, the composition comprises a detectable label, such as a fluorescent dye, a fluorescence quencher, an energy-transfer pair, a quantum dot, or a chemiluminescent precursor. In some embodiments, the label comprises a fluorescein, a rhodamine, or a cyanine. In some embodiments, the label is attached to a second complementary polynucleotide that is hybridized to the D-form polynucleotide sequence portion.

Also provided is an array of different-sequence polynucleotides comprising 5 to 100 L-nucleotides, wherein the polynucleotides are immobilized at addressable locations on a solid support. In some embodiments, the solid support comprises polystyrene, glass, silica gel, silica, polyacrylamide, polyacrylate, hydroxyethylmethacrylate, polyamide, polyethylene, polyethyleneoxy, or nylon. In some embodiments, the solid support comprises a small particle, a bead, a membrane, a frit, a slide, a plate, a micromachined chip, an alkanethiol-gold layer, a non-porous surface, an addressable array, or a gel. In some embodiments, the solid support comprises a bead. In some embodiments, the solid support comprises a polystyrene bead. In some embodiments, the solid support comprises a nylon membrane. In some embodiments, the solid support comprises a small particle selected from a nanoparticle, a microsphere, or a liposome. In some embodiments, the solid support comprises glass, such as contolled pore glass. In some embodiments, the first complementary polynucleotide is attached to the support via a cleavable linker. In some embodiments, the cleavable linker comprises a carbonyl group through which the first complementary polynucleotide is linked to the support. In some embodiments, the solid support is configured as a 96 well format. In some embodiments, at least one polynucleotide comprises a label. In some embodiments, the label comprises a fluorescent dye, a quencher, an energy-transfer dye, a quantum dot, digoxigenin, biotin, a mobility-modifier, a polypeptide, a hybridization-stabilizing moiety, or a chemiluminescent precursor. In some embodiments, at least one immobilized polynucleotide comprises the structure:

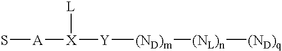

wherein S is a solid support;

A is a linker;

X is a linker with three or more attachment sites;

Y is O, NH, NR, or S, where R is selected from C₁-C₆ alkyl, C₁-C₆ substituted alkyl, C₅-C₁₄ aryl, and C₅-C₁₄ substituted aryl;

L is hydrogen or a label;

N_{L} is a sequence of L-form nucleotides;

N_{D} is a sequence of D-form nucleotides;

m is an integer from 0 to 100; and

n is an integer from 5 to 100; and

q is an integer from 0 to 100.

In some embodiments, A is a cleavable linker. In some embodiments, A comprises one or more of the structures:

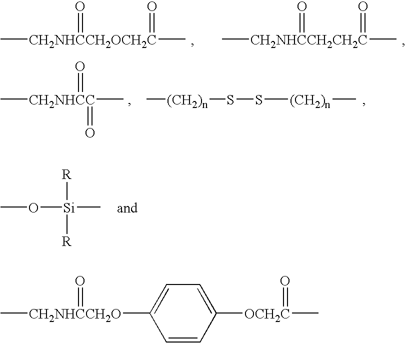

In some embodiments, (N_{D})ₘ and (N_{L})ₙ, and (N_{L})ₙ and (N_{D})_{q}, are linked to each other by linkers. In some embodiments, the linker comprises one or more ethyleneoxy units. In some embodiments, m=0. In some embodiments, m=q=0.

Also provided are various methods. In some embodiments, the invention comprises a method of forming a polynucleotide hybrid comprising providing a heteroconfigurational polynucleotide comprising a D-form polynucleotide sequence portion and an L-form polynucleotide sequence portion that is covalently linked to the D-form polynucleotide sequence portion, and hybridizing the heteroconfigurational polynucleotide to a first complementary polynucleotide to form a duplex between the first complementary polynucleotide and the L-form polynucleotide sequence portion. In some embodiments, the L-form polynucleotide sequence portion comprises 5 to 50 L-nucleotides. In some embodiments, the D-form polynucleotide sequence portion comprises 5 to 50 D-nucleotides. In some embodiments, the L-form polynucleotide sequence portion comprises 5 to 50 L-nucleotides. In some embodiments, the L-form polynucleotide sequence portion comprises at least one L-form 2′-4′ LNA nucleotide. In some embodiments, the L-form polynucleotide sequence portion comprises at least one L-form nucleotide comprising a 1′-α-anomeric nucleotide or a 4′-α-anomeric nucleotide. In some embodiments, the L-form polynucleotide sequence portion comprises at least one L-form nucleotide comprising ribose, arabinose, xylose, or pyranose, in the 1′-β anomeric configuration. In some embodiments, the L-form polynucleotide sequence portion comprises at least one L-form nucleotide comprising ribose, arabinose, xylose, or pyranose, in the 1′-α anomeric configuration. In some embodiments, the L-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose. In some embodiments, the D-form polynucleotide sequence portion comprises at least one D-form 2′-4′ LNA nucleotide. In some embodiments, the D-form polynucleotide sequence portion comprises at least one L-form nucleotide comprising a 1′-α-anomeric nucleotide or a 4′-α-anomeric nucleotide. In some embodiments, the D-form polynucleotide sequence portion comprises at least one L-form nucleotide comprising ribose, arabinose, xylose, or pyranose, in the 1′-β anomeric configuration. In some embodiments, the D-form polynucleotide sequence portion comprises at least one L-form nucleotide comprising ribose, arabinose, xylose, or pyranose, in the 1′-α anomeric configuration. In some embodiments, the D-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose. In some embodiments, at least one of the D-form polynucleotide sequence portion and the L-form polynucleotide sequence portion comprises an internucleotide linkage selected from a 2-aminoethylglycine, a phosphorothioate, a phosphorodithioate, a phosphotriester, and a phosphoramidate. In some embodiments, the first complementary polynucleotide comprises at least one L-form nucleotide. In some embodiments, the first complementary polynucleotide comprises at least one L-form 2′ deoxyribose or 2′-4′ LNA nucleotide. In some embodiments, the first complementary polynucleotide comprises at least two peptide nucleic acid subunits. In some embodiments, unhybridized first complementary polynucleotide is separated from said hybrid. In some embodiments, the method comprises detecting the hybrid. In some embodiments, The method comprises primer extension of the heteroconfigurational polynucleotide. In some embodiments, the method comprises cleavage of the heteroconfigurational polynucleotide by a nuclease enzyme. In some embodiments, the method comprises ligation of a heteroconfigurational polynucleotide to a polynucleotide that is hybridized adjacent to an end of the heteroconfigurational polynucleotide. In some embodiments, the hybrid is immobilized on a solid support.

Also provided are kits. In some embodiments, the kit comprises a heteroconfigurational polynucleotide as above, and a solid support to which is attached at least one polynucleotide comprising an L-form polynucleotide sequence portion that is complementary to the L-form polynucleotide sequence portion in the heteroconfigurational polynucleotide. In some embodiments, the kit comprises a plurality of solid supports, each support being attached to a heteroconfigurational polynucleotide comprising an L-form polynucleotide sequence portion comprising a unique sequence that is distinct from the sequences of the L-form polynucleotide sequence portions in the other solid supports of said plurality. In some embodiments, the kit comprises an addressable array of heteroconfigurational polynucleotide at different locations, each polynucleotide comprising an L-form heteroconfigurational polynucleotide sequence portion comprising a unique sequence that is distinct from the sequences of the L-form polynucleotide sequence portions in the heteroconfigurational polynucleotides at other locations on the array. In some embodiments, the kit comprises at least 10 different heteroconfigurational polynucleotides each comprising a unique sequence that is distinct from the L-form polynucleotide sequence portions in the other heteroconfigurational polynucleotides. In some embodiments, the kit comprises at least 100 different heteroconfigurational polynucleotides each comprising a unique sequence that is distinct from the L-form polynucleotide sequence portions in the other heteroconfigurational polynucleotides.

These and other features of the invention will become more apparent from the Drawings and the following description.

### BRIEF DESCRIPTION OF THE DRAWINGS

FIG. 1 shows a D-form DNA portion of an oligonucleotide and the mirror image L-form DNA portion of the oligonucleotide.

FIG. 2 shows hybridization of a heteroconfigurational oligonucleotide with a target polynucleotide and primer extension of the heteroconfigurational oligonucleotide/target hybrid.

FIG. 3 shows exemplary embodiments of a labelled heteroconfigurational oligonucleotide/target hybrid where (a) the terminus of the L-form sequence portion is covalently attached to a label, (b) the D-form sequence portion is covalently attached to a label, (c) the target is multiply labelled, and (d) labels are incorporated by primer extension with labelled nucleotide 5′-triphosphates.

FIG. 4 shows ligation of a heteroconfigurational oligonucleotide probe and a second probe.

FIG. 5 shows a PCR with a heteroconfigurational oligonucleotide primer to form an L-form sequence tagged amplicon.

FIG. 6 shows an addressable array of L-form sequence containing, immobilized oligonucleotides. Each location, represented by a circle ◯, may comprise a unique L-form sequence. The L-form sequence can hybridize to the complementary L-form sequence of a heteroconfigurational oligonucleotide.

FIG. 7 shows a probe labelled with a fluorescent dye (F) and a quencher (Q) whereby fluorescence is quenched by proximity to the quencher in the non-hybridized state (left). Upon hybridization to a target sequence, the fluorescent dye and quencher are physically separated sufficiently to allow fluorescence.

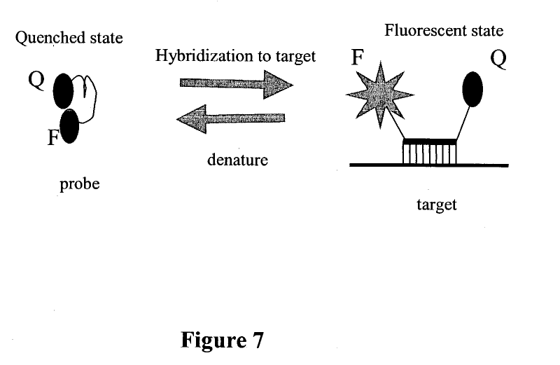

FIG. 8 shows an exemplary ligation reaction followed by PCR amplification.

FIG. 9 shows exemplary embodiments of immobilized labelled hybrids on an addressable array.

FIG. 10 shows an exemplary embodiment of an immobilized labelled hybrid where multiple nucleotides of the target sequence are labelled and a location may be labelled as a control.

FIG. 11 shows primer extension of a heteroconfigurational oligonucleotide/target hybrid with a labelled dideoxynucleotide 5′-triphosphate at an SNP site (X). The extended hybrid may be denatured and the extended primer may be separated from the target, purified and detected.

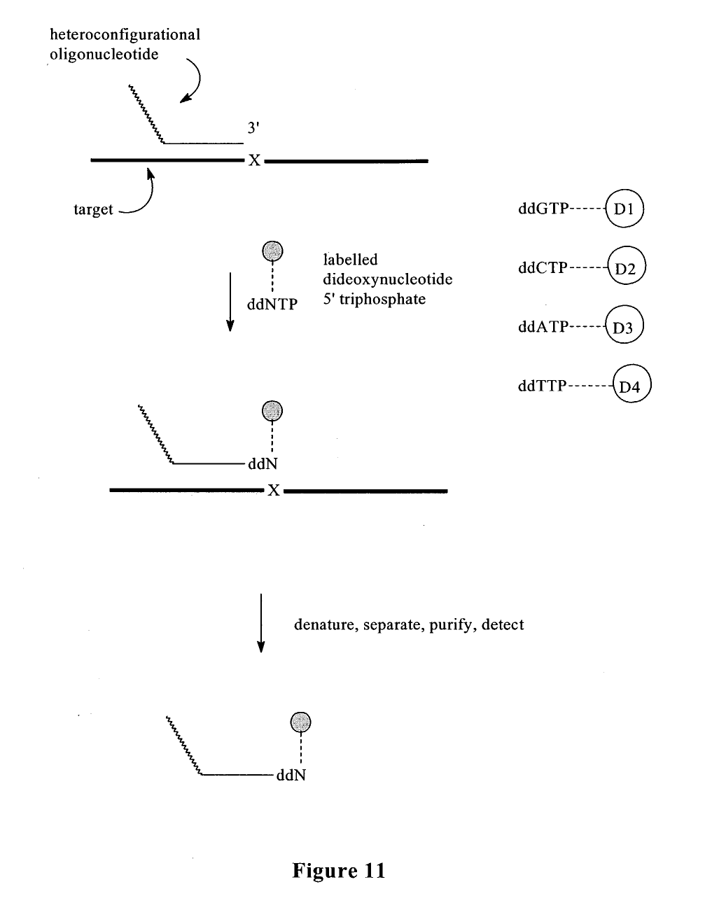

FIG. 12 shows a quantitative, three-dimensional plot of the averaged fluorescent intensities of the hybridizations on spotted arrays.

### DETAILED DESCRIPTION

Reference will now be made in detail to certain embodiments of the invention, examples of which are illustrated in the accompanying Examples. While the invention will be described in conjunction with the exemplary embodiments, it will be understood that they are not intended to limit the invention to those embodiments. On the contrary, the invention is intended to cover all alternatives, modifications, and equivalents which may be included within the scope of the invention.

Definitions

Stereochemical terms are used in accordance with: “Sterochemistry of Organic Compounds” (1994) E. Eliel and S. Wilen, John Wiley & Sons, Inc., New York.

The term “configuration” refers to the spatial array of atoms that distinguishes stereoisomers (isomers of the same constitution) other than distinctions due to differences in conformation. Configurational isomers are stereoisomers that differ in configuration. Absolute configurations of the novel compositions herein are defined by their particular chiral centers (e.g. sugar carbon atoms). The chiral carbons are designated by means of alphabetic symbols for rotation: R for rectus and S for sinister) defined by the bond priority rules of Cahn, Ingold, and Prelog (“Organic Chemistry”, Fifth Edition (2000) J. McMurry, Brooks/Cole, Pacific Grove, Calif., pp. 315-319).

The term “heteroconfigurational” refers to a compound with subunits comprising different stereochemical configurations.

“Nucleobase” means any nitrogen-containing heterocyclic moiety capable of forming Watson-Crick hydrogen bonds in pairing with a complementary nucleobase or nucleobase analog, e.g. a purine, a 7-deazapurine, or a pyrimidine. Typical nucleobases are the naturally occurring nucleobases adenine, guanine, cytosine, uracil, thymine, and analogs (Seela, U.S. Pat. No. 5,446,139) of the naturally occurring nucleobases, e.g. 7-deazaadenine, 7-deazaguanine, 7-deaza-8-azaguanine, 7-deaza-8-azaadenine, inosine, nebularine, nitropyrrole (Bergstrom, (1995) J. Amer. Chem. Soc. 117:1201-09), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121; Gall, WO 01/38584), and ethenoadenine (Fasman (1989) in Practical Handbook of Biochemistry and Molecular Biology, pp. 385-394, CRC Press, Boca Raton, Fla.).

“Nucleoside” refers to a compound consisting of a nucleobase linked to the C-1′ carbon of a sugar, such as ribose, arabinose, xylose, and pyranose, in the natural β or the α anomeric configuration. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Ribose examples include ribose, 2′-deoxyribose, 2′,3′-dideoxyribose, 2′-haloribose, 2′-fluororibose, 2′-chlororibose, and 2′-alkylribose, e.g. 2′-O-methyl, 4′-α-anomeric nucleotides, 1′-α-anomeric nucleotides (Asseline (1991) Nucl. Acids Res. 19:4067-74), 2′-4′- and 3′-4′-linked and other “locked” or “LNA”, bicyclic sugar modifications (WO 98/22489; WO 98/39352; WO 99/14226). Exemplary LNA sugar analogs within a polynucleotide include the structures:

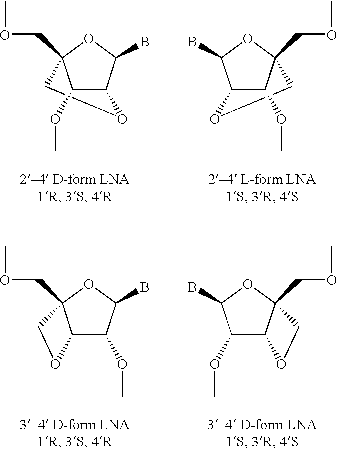

where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleobase is purine, e.g. A or G, the ribose sugar is usually attached to the N⁹-position of the nucleobase. When the nucleobase is pyrimidine, e.g. C, T or U, the pentose sugar is usually attached to the N¹-position of the nucleobase (Kornberg and Baker, (1992) DNA Replication, 2^{nd} Ed., Freeman, San Francisco, Calif.).

“Nucleotide” refers to a phosphate ester of a nucleoside, as a monomer unit or within a nucleic acid. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides have associated counter ions, such as H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. Polynucleotides may be comprised of nucleobase and sugar analogs. Polynucleotides typically range in size from a few monomeric units, e.g. 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted.

The term “heteroconfigurational oligonucleotide” means an oligonucleotide comprising nucleotides of different configurations. Heteroconfigurational oligonucleotides have one or more portions of L-form nucleotides and one or more portions of D-form nucleotides.

“Internucleotide analog” means a phosphate ester analog or a non-phosphate analog of a polynucleotide. Phosphate ester analogs include: (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Non-phosphate analogs include compounds wherein the sugar/phosphate moieties are replaced by an amide linkage, such as a 2-aminoethylglycine unit, commonly referred to as PNA (Buchardt, WO 92/20702; Nielsen (1991) Science 254:1497-1500).

“Polypeptide” refers to a polymer including proteins, synthetic peptides, antibodies, peptide analogs, and peptidomimetics in which the monomers are amino acids and are joined together through amide bonds. When the amino acids are α-amino acids, either the L-optical isomer or the D-optical isomer can be used. Additionally, unnatural amino acids, for example, valanine, phenylglycine and homoarginine are also included. Commonly encountered amino acids that are not gene-encoded may also be used in the present invention. All of the amino acids used in the present invention may be either the D- or L-optical isomer. In addition, other peptidomimetics are also useful in the present invention. For a general review, see Spatola, A. F., in Chemistry and Biochemistry of Amino Acids, Peptides and Proteins, B. Weinstein, eds., Marcel Dekker, New York, p. 267 (1983).

The term “amino acid” refers to naturally occurring and synthetic amino acids, as well as amino acid analogs that contain an amino group and a carboxylic acid group.

“Attachment site” refers to a site on a moiety or a molecule, e.g. a quencher, a fluorescent dye, or a polynucleotide, to which is covalently attached, or capable of being covalently attached, a linker or another moiety.

“Linker” refers to a chemical moiety in a molecule comprising a covalent bond or a chain of atoms that covalently attaches one moiety or molecule to another, e.g. a quencher to a polynucleotide. A “cleavable linker” is a linker which has one or more covalent bonds which may be broken by the result of a reaction or condition. For example, an ester in a molecule is a linker that may be cleaved by a reagent, e.g. sodium hydroxide, resulting in a carboxylate-containing fragment and a hydroxyl-containing product

“Reactive linking group” refers to a chemically reactive substituent or moiety, e.g. a nucleophile or electrophile, on a molecule which is capable of reacting with another molecule to form a covalent bond. Reactive linking groups include active esters, which are commonly used for coupling with amine groups. For example, N-hydroxysuccinimide (NHS) esters have selectivity toward aliphatic amines to form aliphatic amide products which are very stable. Their reaction rate with aromatic amines, alcohols, phenols (tyrosine), and histidine is relatively low. Reaction of NHS esters with amines under nonaqueous conditions is facile, so they are useful for derivatization of small peptides and other low molecular weight biomolecules. Virtually any molecule that contains a carboxylic acid or that can be chemically modified to contain a carboxylic acid can be converted into its NHS ester. NHS esters are available with sulfonate groups that have improved water solubility.

“Substituted” as used herein refers to a molecule wherein one or more hydrogen atoms are replaced with one or more non-hydrogen atoms, functional groups or moieties. For example, an unsubstituted nitrogen is —NH₂, while a substituted nitrogen is —NHCH₃. Exemplary substituents include but are not limited to halo, e.g., fluorine and chlorine, C₁-C₈ alkyl, sulfate, sulfonate, sulfone, amino, ammonium, amido, nitrile, nitro, alkoxy (—OR where R is C₁-C₁₂ alkyl), phenoxy, aromatic, phenyl, polycyclic aromatic, heterocycle, water-solubilizing group, and linking moiety.

“Alkyl” means a saturated or unsaturated, branched, straight-chain, branched, cyclic, or substituted hydrocarbon radical derived by the removal of one hydrogen atom from a single carbon atom of a parent alkane, alkene, or alkyne. Typical alkyl groups consist of 1-12 saturated and/or unsaturated carbons, including, but not limited to, methyl, ethyl, cyanoethyl, isopropyl, butyl, and the like.

“Alkyldiyl” means a saturated or unsaturated, branched, straight chain, cyclic, or substituted hydrocarbon radical of 1-12 carbon atoms, and having two monovalent radical centers derived by the removal of two hydrogen atoms from the same or two different carbon atoms of a parent alkane, alkene or alkyne. Typical alkyldiyl radicals include, but are not limited to, 1,2-ethyldiyl (—CH₂CH₂—), 1,3-propyldiyl (—CH₂CH₂CH₂—), 1,4-butyldiyl (—CH₂CH₂CH₂CH₂—), and the like. “Alkoxydiyl” means an alkoxyl group having two monovalent radical centers derived by the removal of a hydrogen atom from the oxygen and a second radical derived by the removal of a hydrogen atom from a carbon atom. Typical alkoxydiyl radicals include, but are not limited to, methoxydiyl (—OCH₂—) and 1,2-ethoxydiyl or ethyleneoxy (—OCH₂CH₂—). “Alkylaminodiyl” means an alkylamino group having two monovalent radical centers derived by the removal of a hydrogen atom from the nitrogen and a second radical derived by the removal of a hydrogen atom from a carbon atom. Typical alkylaminodiyl radicals include, but are not limited to —NHCH₂—, —NHCH₂CH₂—, and —NHCH₂CH₂CH₂—. “Alkylamidediyl” means an alkylamide group having two monovalent radical centers derived by the removal of a hydrogen atom from the nitrogen and a second radical derived by the removal of a hydrogen atom from a carbon atom. Typical alkylamidediyl radicals include, but are not limited to —NHC(O)CH₂—, —NHC(O)CH₂CH₂—, and —NHC(O)CH₂CH₂CH₂—.

“Aryl” means a monovalent aromatic hydrocarbon radical of 5-14 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like, including substituted aryl groups.

“Aryldiyl” means an unsaturated cyclic or polycyclic hydrocarbon radical of 5-14 carbon atoms having a conjugated resonance electron system and at least two monovalent radical centers derived by the removal of two hydrogen atoms from two different carbon atoms of a parent aryl compound, including substituted aryldiyl groups.

“Substituted alkyl”, “substituted alkyldiyl”, “substituted aryl” and “substituted aryldiyl” mean alkyl, alkyldiyl, aryl and aryldiyl respectively, in which one or more hydrogen atoms are each independently replaced with another substituent. Typical substituents include, but are not limited to, F, Cl, Br, I, R, OH, —OR, —SR, SH, NH₂, NHR, NR₂, —⁺NR₃, —N═NR₂, —CX₃, —CN, —OCN, —SCN, —NCO, —NCS, —NO, —NO₂, —N₂⁺, —N₃, —NHC(O)R, —C(O)R, —C(O)NR₂—S(O)₂O⁻, —S(O)₂R, —OS(O)₂OR, —S(O)₂NR, —S(O)R, —OP(O)(OR)₂, —P(O)(OR)₂, —P(O)(O—)₂, —P(O)(OH)₂, —C(O)R, —C(O)X, —C(S)R, —C(O)OR, —CO₂⁻, —C(S)OR, —C(O)SR, —C(S)SR, —C(O)NR₂, —C(S)NR₂, —C(NR)NR₂, where each R is independently —H, C₁-C₆ alkyl, C₅-C₁₄ aryl, heterocycle, or linking group. Substituents also include divalent, bridging functionality, such as diazo (—N═N—), ester, ether, ketone, phosphate, alkyldiyl, and aryldiyl groups.

“Heterocycle” refers to a molecule with a ring system in which one or more ring atoms is a heteroatom, e.g. nitrogen, oxygen, and sulfur (as opposed to carbon).

“Enzymatically extendable” refers to a nucleotide which is: (i) capable of being enzymatically incorporated onto a terminus of a polynucleotide through the action of a polymerase enzyme, and (ii) capable of supporting further primer extension. Enzymatically extendable nucleotides include nucleotide 5′-triphosphates, i.e. dNTP and NTP, and labelled forms thereof.

“Enzymatically incorporatable” refers to a nucleotide which is capable of being enzymatically incorporated onto a terminus of a polynucleotide through the action of a polymerase enzyme. Enzymatically incorporatable nucleotides include dNTP, NTP, and 2′,3′-dideoxynucleotide 5′-triphosphates, i.e. ddNTP, and labelled forms thereof.

“Terminator nucleotide” means a nucleotide which is capable of being enzymatically incorporated onto a terminus of a polynucleotide through the action of a polymerase enzyme, but is then cannot be further extended, i.e. a terminator nucleotide is enzymatically incorporatable, but not enzymatically extendable. Examples of terminator nucleotides include ddNTP and 2′-deoxy, 3′-fluoro nucleotide 5′-triphosphates, and labelled forms therof.

“Target”, “target polynucleotide”, and “target sequence” mean a specific polynucleotide sequence, the presence or absence of which is to be detected, and that is the subject of hybridization with a complementary polynucleotide, e.g. a primer or probe. The target sequence can be composed of DNA, RNA, an analog thereof, and including combinations thereof. The target can be single-stranded or double-stranded. In primer extension processes, the target polynucleotide which forms a hybridization duplex with the primer may also be referred to as a “template.” A template serves as a pattern for the synthesis of another, complementary nucleic acid (Concise Dictionary of Biomedicine and Molecular Biology, (1996) CPL Scientific Publishing Services, CRC Press, Newbury, UK). A target sequence for use with the present invention may be derived from any living, or once living, organism, including but not limited to prokaryote, eukaryote, plant, animal, and virus. The target sequence may originate from a nucleus of a cell, e.g., genomic DNA, or may be extranuclear nucleic acid, e.g., plasmid, mitrochondrial nucleic acid, various RNAs, and the like. The target nucleic acid sequence may be first reverse-transcribed into cDNA if the target nucleic acid is RNA. A variety of methods are available for obtaining a target nucleic acid sequence for use with the compositions and methods of the present invention. When the target sequence is obtained through isolation from a biological sample, preferred isolation techniques include (1) organic extraction followed by ethanol precipitation, e.g., using a phenol/chloroform organic reagent (e.g., Ausubel et al., eds., (1993) Current Protocols in Molecular Biology Volume 1, Chapter 2, Section I, John Wiley & Sons, New York), or an automated DNA extractor (e.g., Model 341 DNA Extractor, Applied Biosystems, Foster City, Calif.); (2) stationary phase adsorption methods (e.g., Boom et al., U.S. Pat. No. 5,234,809; Walsh et al., (1991) Biotechniques 10(4): 506-513); and (3) salt-induced DNA precipitation methods (e.g., Miller et al., (1988) Nucleic Acids Research, 16(3): 9-10).

The term “probe” means a polynucleotide that is capable of forming a duplex structure by complementary base pairing with a sequence of a target polynucleotide. For example, probes may be labelled, e.g. with a quencher moiety, or an energy transfer pair comprised of a fluorescent reporter and quencher.

“Primer” means an oligonucleotide of defined sequence that is designed to hybridize with a complementary, primer-specific portion of a target sequence, a probe, or a ligation product, and undergo primer extension. A primer functions as the starting point for the polymerization of nucleotides (Concise Dictionary of Biomedicine and Molecular Biology, (1996) CPL Scientific Publishing Services, CRC Press, Newbury, UK).

The terms “duplex” means an intermolecular or intramolecular double-stranded portion of a nucleic acid which is base-paired through Watson-Crick, Hoogsteen, or other sequence-specific interactions of nucleobases. A duplex may consist of a primer and a template strand, or a probe and a target strand. A “hybrid” means a duplex, triplex, or other base-paired complex of nucleic acids interacting by base-specific interactions, e.g. hydrogen bonds.

The term “primer extension” means the process of elongating a primer that is annealed to a target in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand.

The term “label” refers to any moiety which can be attached to a polynucleotide and: (i) provides a detectable signal; (ii) interacts with a second label to modify the detectable signal provided by the second label, e.g. FRET; (iii) stabilizes hybridization, i.e. duplex formation; (iv) confers a capture function, i.e. hydrophobic affinity, antibody/antigen, ionic complexation, or (v) changes a physical property, such as electrophoretic mobility, hydrophobicity, hydrophilicity, solubility, or chromatographic behavior. Labelling can be accomplished using any one of a large number of known techniques employing known labels, linkages, linking groups, reagents, reaction conditions, and analysis and purification methods. Labels include light-emitting or light-absorbing compounds which generate or quench a detectable fluorescent, chemiluminescent, or bioluminescent signal (Kricka, L. in Nonisotopic DNA Probe Techniques (1992), Academic Press, San Diego, pp. 3-28). Fluorescent reporter dyes useful for labelling biomolecules include fluoresceins (for example, U.S. Pat. Nos. 5,188,934; 5,654,442; 6,008,379; 6,020,481), rhodamines (for example, U.S. Pat. Nos. 5,366,860; 5,847,162; 5,936,087; 6,051,719; 6,191,278), benzophenoxazines (for example, U.S. Pat. No. 6,140,500), energy-transfer dye pairs of donors and acceptors (for example, U.S. Pat. Nos. 5,863,727; 5,800,996; 5,945,526), and cyanines (for example, Kubista, WO 97/45539), as well as any other fluorescent label capable of generating a detectable signal. Specific examples of fluorescein dyes include 6-carboxyfluorescein; 2′,4′,1,4,-tetrachlorofluorescein; and 2′,4′,5′,7′,1,4-hexachlorofluorescein (e.g., U.S. Pat. No. 5,654,442). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (Andrus, “Chemical methods for 5′ non-isotopic labelling of PCR probes and primers” (1995) in PCR 2: A Practical Approach, Oxford University Press, Oxford, pp. 39-54). Non-radioactive labelling methods, techniques, and reagents are reviewed in: Non-Radioactive Labelling, A Practical Introduction, Garman, A. J. (1997) Academic Press, San Diego.

As used herein, “energy transfer” refers to the process by which the excited state energy of an excited group, e.g. fluorescent reporter dye, is conveyed through space or through bonds to another group, e.g. a quencher moiety, which may attenuate (quench) or otherwise dissipate or transfer the energy. Energy transfer can occur through fluorescence resonance energy transfer, direct energy transfer, and other mechanisms. The exact energy transfer mechanisms is not limiting to the present invention. It is to be understood that any reference to energy transfer in the instant application encompasses all of these mechanistically-distinct phenomena.

“Energy transfer pair” refers to any two moieties that participate in energy transfer. Typically, one of the moieties acts as a fluorescent reporter, i.e. donor, and the other acts as a fluorescence quencher, i.e. acceptor (“Fluorescence resonance energy transfer.” Selvin P. (1995) Methods Enzymol 246:300-334; dos Remedios C. G. (1995) J. Struct. Biol. 115:175-185; “Resonance energy transfer: methods and applications.” Wu P. and Brand L. (1994) Anal Biochem 218:1-13). Fluorescence resonance energy transfer (FRET) is a distance-dependent interaction between two moieties in which excitation energy, i.e. light, is transferred from a donor (“reporter”) to an acceptor without emission of a photon. The acceptor may be fluorescent and emit the transferred energy at a longer wavelength, or it may be non-fluorescent and serve to diminish the detectable fluorescence of the reporter (quenching). FRET may be either an intermolecular or intramolecular event, and is dependent on the inverse sixth power of the separation of the donor and acceptor, making it useful over distances comparable with the dimensions of biological macromolecules. Thus, the spectral properties of the energy transfer pair as a whole change in some measurable way if the distance between the moieties is altered by some detectable amount. Self-quenching probes incorporating fluorescent donor-nonfluorescent acceptor combinations have been developed primarily for detection of proteolysis (Matayoshi, (1990) Science 247:954-958) and nucleic acid hybridization (“Detection of Energy Transfer and Fluorescence Quenching” Morrison, L., in Nonisotopic DNA Probe Techniques, L. Kricka, Ed., Academic Press, San Diego, (1992) pp. 311-352; Tyagi S. (1998) Nat. Biotechnol. 16:49-53; Tyagi S. (1996) Nat. Biotechnol 14:303-308). In most applications, the donor and acceptor dyes are different, in which case FRET can be detected by the appearance of sensitized fluorescence of the acceptor or by quenching of donor fluorescence.

The term “quenching” refers to a decrease in fluorescence of a fluorescent reporter moiety caused by a quencher moiety by energy transfer, regardless of the mechanism. Hence, illumination of the fluorescent reporter in the presence of the quencher leads to an emission signal that is less intense than expected, or even completely absent.

The terms “annealing” and “hybridizing” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “solid support” refers to any solid phase material upon which an oligonucleotide is synthesized, attached or immobilized. Solid support encompasses terms such as “resin”, “solid phase”, and “support”. A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

“Array” or “microarray” means a predetermined spatial arrangement of polynucleotides present on a solid support or in an arrangement of vessels. Certain array formats are referred to as a “chip” or “biochip” (M. Schena, Ed. Microarray Biochip Technology, BioTechnique Books, Eaton Publishing, Natick, Mass. (2000). An array can comprise a low-density number of addressable locations, e.g. 2 to about 12, medium-density, e.g. about a hundred or more locations, or a high-density number, e.g. a thousand or more. Typically, the array format is a geometrically-regular shape which allows for fabrication, handling, placement, stacking, reagent introduction, detection, and storage. The array may be configured in a row and column format, with regular spacing between each location. Alternatively, the locations may be bundled, mixed, or homogeneously blended for equalized treatment or sampling. An array may comprise a plurality of addressable locations configured so that each location is spatially addressable for high-throughput handling, robotic delivery, masking, or sampling of reagents, or by detection means including scanning by laser illumination and confocal or deflective light gathering.

The term “end-point analysis” refers to a method where data collection occurs only when a reaction is substantially complete.

The term “real-time analysis” refers to periodic monitoring during PCR. Certain systems such as the ABI 7700 and 7900HT Sequence Detection Systems (Applied Biosystems, Foster City, Calif.) conduct monitoring during each thermal cycle at a pre-determined or user-defined stage in each cycle. Real-time analysis of PCR with FRET probes measures fluorescent dye signal changes from cycle-to-cycle, preferably minus any internal control signals.

Exemplary Heteroconfigurational Oligonucleotide Compositions

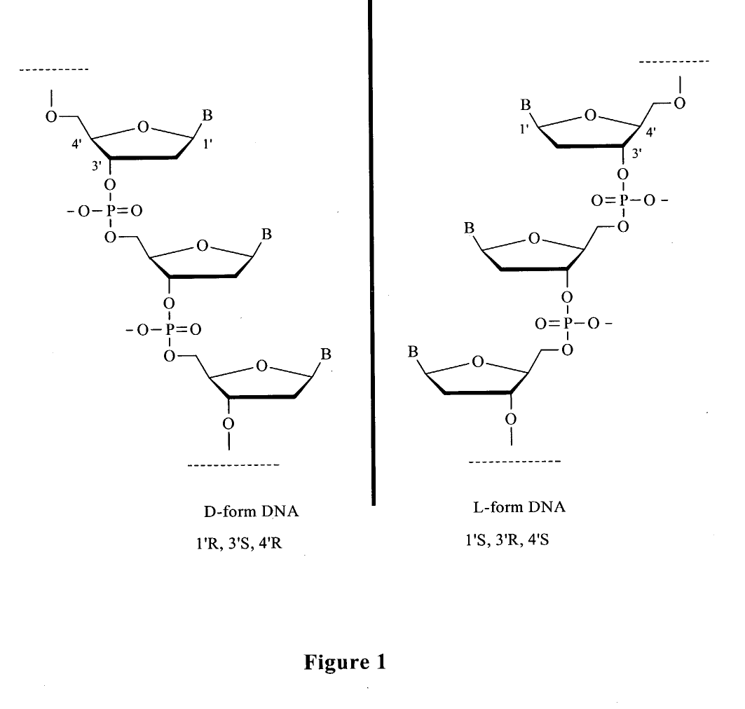

In some embodiments, compositions of the invention include heteroconfigurational oligonucleotides which have many uses, such as in molecular biology and nucleic acid-based diagnostic assays. Heteroconfigurational oligonucleotides are oligonucleotides that comprise at least one L-form (L-configuration nucleotides) sequence portion attached to at least one D-form (D-configuration nucleotides) sequence portion. The sequence portions may be linked to each other by any means, typically by a bond or a linker. In some embodiments, a D-form sequence portion contains at least five D-nucleotides so as to form a stable duplex by hybridization to its L-form sequence complement. In some embodiments, a heteroconfigurational oligonucleotide includes an L-form sequence portion comprising 5 to 50 L-nucleotides covalently attached by a bond or a linker to a D-form sequence portion comprising 5 to 50 D-nucleotides. The L-configuration of the sugar moiety of compounds of the present invention contrasts with the D-configuration of ribose sugar moieties of most naturally occurring nucleosides such as cytidine, adenosine, thymidine, guanosine and uridine. The L-configuration of the sugars are defined by the chirality at the 1′, 3′, and 4′ carbon atoms, as well as the 2′ for ribose carbon atoms. L-form nucleotides are the mirror image, enantiomeric stereoisomer of the naturally-occurring D-form nucleotides. FIG. 1 shows mirror image D-form and L-form portions of a DNA oligonucleotide. The absolute configurations are noted at the 1′, 3′, and 4′ asymmetric, chiral carbon positions. RNA has an additional chiral carbon at the 2′ position.

In some embodiments, the invention includes a labelled heteroconfigurational oligonucleotide that comprises at least one label. Typically, a label can be linked covalently to heteroconfigurational oligonucleotides by a bond or a linker. Labels may be as defined above, such as a fluorescent dye, a quencher, an energy-transfer dye, a quantum dot, digoxigenin, biotin, a mobility-modifier, a polypeptide, a hybridization-stabilizing moiety, or a chemiluminescent precursor. Exemplary fluorescent dye labels include compounds from the fluorescein, rhodamine, and cyanine structural types, exemplified by the structures:

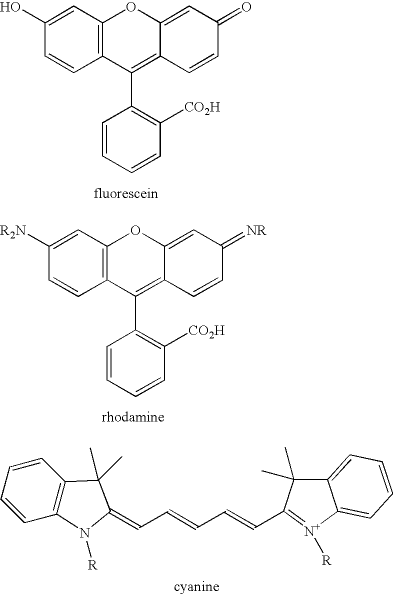

Quencher labels undergo energy transfer of fluorescence emitted from fluorescent dyes by the intramolecular fluorescence resonance energy transfer (FRET) effect. Quenchers may themselves be fluorescent or non-fluorescent (for example, see Reed, WO 01/42505; and Cook, WO 00/75378). Quencher labels include compounds selected from the fluorescein, rhodamine, nitro-cyanine (Lee, U.S. Pat. No. 6,080,868), and aryl-diazo structural types, for example.

A label can also comprise a hybridization-stabilizing moiety, such as a minor groove binder, intercalator, polycation, such as polylysine and spermine, or a cross-linking functional group. Hybridization-stabilizers may increase the stability of base-pairing, i.e. affinity, or the rate of hybridization (Corey (1995) J. Amer. Chem. Soc. 117:9373-74) of the primer and target, or probe and target. Hybridization-stabilizers serve to increase the specificity of base-pairing, exemplified by large differences in Tm between perfectly complementary oligonucleotide and target sequences and where the resulting duplex contains one or more mismatches of Watson/Crick base-pairing (Blackburn, G. and Gait, M. Eds. “DNA and RNA structure” in Nucleic Acids in Chemistry and Biology, 2^{nd} Edition, (1996) Oxford University Press, pp. 15-81 and 337-46). Exemplary minor groove binders include Hoechst 33258 (Rajur (1997) J. Org. Chem. 62:523-29), distamycin, netropsin, (Gong (1997) Biochem. and Biophys. Res. Comm. 240:557-60), and CDPI₁₋₃ (U.S. Pat. No. 5,801,155; WO 96/32496). An example of a minor groove binder is CDPI₃, represented by the structure:

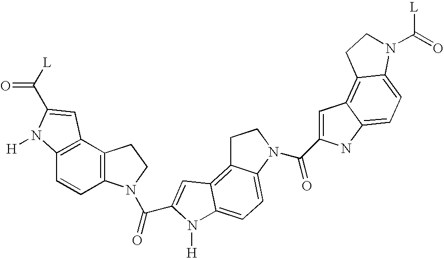

where L are the sites of attachment to a heteroconfigurational oligonucleotide (Dempcy, WO 01/31063).

When the linker to the label is attached to a nucleobase of a heteroconfigurational oligonucleotide, the nucleobase attachment site is usually at the 8-position of a purine nucleobase, the 7- or 8-position of a 7-deazapurine nucleobase, and the 5-position of a pyrimidine nucleobase, although other attachment sites may also be used. The linker to the label may be any alkyldiyl or aryldiyl linker, or substituted form thereof, including the structures:

B—C≡C—CH₂(OCH₂CH₂)ₘNR¹-L

B—C≡C—CH₂(OCH₂CH₂)ₘNR¹—X-L

where B is a nucleobase; L is a label; R¹ is H or (C₁-C₈) alkyl; and m is 0, 1, or 2 (Khan, U.S. Pat. Nos. 5,770,716 and 5,821,356; Hobbs, U.S. Pat. No. 5,151,507). X is an amide substructure, including the exemplary structures:

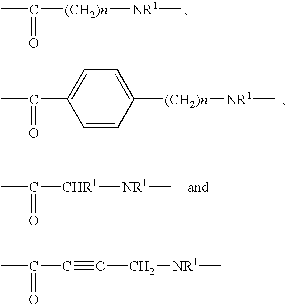

where n is an integer from 1 to 5.

A labelled heteroconfigurational oligonucleotide may have a label attached through a nucleobase. An exemplary embodiment is structure I:

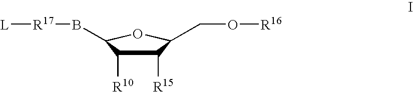

where L is a label; B is a nucleobase, including uracil, thymine, cytosine, adenine, 7-deazaadenine, guanine, and 7-deazaguanosine; R¹⁰ is H, OH, halide, azide, amine, alkylamine, alkyl (C₁-C₆), allyl, alkoxy (C₁-C₆), OCH₃, or OCH₂CH═CH₂; R¹⁵ is H, phosphate, internucleotide phosphodiester, or internucleotide analog; R¹⁶ is H, phosphate, internucleotide phosphodiester, or internucleotide analog; and R¹⁷ is a bond or linker. An exemplary linker comprising a propargyl or vinylic group is shown immediately below:

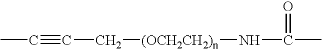

where n is 0, 1, or 2.

Alternatively, a labelled heteroconfigurational oligonucleotide may have a label attached at a 5′ terminus. An exemplary embodiment is structure II:

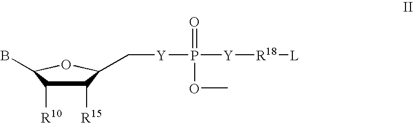

where L, B, R¹⁰ and R¹⁵ are selected as from structure I. Each Y is independently O, NH, NR, or S, where R is selected from C₁-C₆ alkyl, C₁-C₆ substituted alkyl, C₅-C₁₄ aryl, and C₅-C₁₄ substituted aryl. R¹⁸ may be a bond or any covalent linker for attaching the 5′ phosphate, or phosphate analog, of the heteroconfigurational oligonucleotide and the label. For example, R¹⁸ may be a chain of 1-100 ethyleneoxy (also called polyethyleneoxy or PEO) units, —(CH₂CH₂O)ₙ—, where n is 1 to 100), C₁-C₁₂ alkyldiyl, C₁-C₁₂ substituted alkyldiyl; C₅-C₁₄ aryldiyl, or C₅-C₁₄ substituted aryldiyl. An exemplary embodiment of R¹⁸ is shown immediately below:

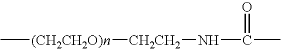

where n ranges from 1 to 10.

Alternatively, a labelled heteroconfigurational oligonucleotide may have a label attached at a 3′ terminus. An exemplary embodiment is structure III:

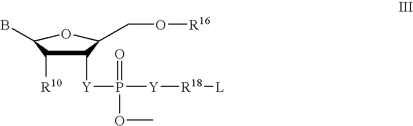

where L, Y, B, R¹⁰, R¹⁶ and R¹⁸ are as defined for structures I and II above.

Labelled heteroconfigurational oligonucleotide may comprise more than one label. One embodiment of a heteroconfigurational oligonucleotide comprises an energy transfer pair including a reporter dye and a quencher whereby fluorescence energy transfer can occur between the reporter dye and quencher. The reporter dye may be any suitable dye, such as a fluorescein, a rhodamine, a dioxetane chemiluminescent dye, a coumarin, a naphthylamine, a cyanine or a bodipy dye.

Typically, the reporter dye is attached to the heteroconfigurational oligonucleotide by a first linkage and the quencher is attached to the heteroconfigurational oligonucleotide by a second linkage. The reporter dye and the quencher are oriented such that when the labelled heteroconfigurational oligonucleotide is hybridized to a target polynucleotide sequence the reporter dye is not fully quenched by the quencher, and when the labelled oligonucleotide is not hybridized to a target polynucleotide sequence the reporter dye is effectively quenched by the quencher.

In some embodiments, the reporter dye and quencher labels are covalently attached at the termini of the heteroconfigurational oligonucleotide. For example, either the reporter dye or the quencher is attached at the 3′end and the other is attached at the 5′-end.

The nucleotide sequence of a reporter/quencher heteroconfigurational oligo-nucleotide may be selected to contain sufficient self-complementarity to form a stable hairpin structure, due to the presence of complementary L-form DNA sequence portions that flank a target-complementary D-form sequence portion and that form a duplex when the heteroconfigurational oligonucleotide is not hybridized to a complementary target sequence. In this embodiment, the reporter and quencher moieties can be located at distal ends of each L-form sequence portion, such that the reporter and quencher moieties are in close proximity when the hairpin-structure is formed, and are far apart when the inner D-form sequence portion is hybridized to a complementary target sequence. The thermal melting properties (Tm) of the hairpin-forming reporter/quencher heteroconfigurational oligonucleotide may be optimized by sequence design such that in the absence of the complementary target sequence, fluorescence from the reporter is effectively quenched by the quencher whereas in the presence of the complementary target sequence and upon formation of a hybridization duplex, quenching is precluded, or is substantially and measurably precluded, while fluorescence increases. By this effect, the presence of a specific target sequence in a sample may be detected, and in some instances, quantitated. When the target sequence is within a PCR amplicon, PCR may be monitored and detected.

In some embodiments, the present invention includes heteroconfigurational oligonucleotides labelled with an energy-transfer pair comprising a donor and an acceptor. The donor dye absorbs light at a first wavelength and emits excitation energy. The acceptor dye is capable of absorbing the excitation energy emitted by the donor dye and fluorescing at a second wavelength in response. Energy-transfer pairs have advantages for use in the simultaneous detection of multiple labelled substrates in a mixture, such as DNA sequencing. A single donor dye can be used in a set of energy-transfer dyes so that each dye has strong absorption at a common wavelength. By then varying the acceptor dye in the energy-transfer set, the acceptor dyes can be spectrally resolved by their respective emission maxima.

The donor dye may be attached to the acceptor dye through a linker that facilitates efficient energy transfer between the donor and acceptor dyes (e.g., see Lee, U.S. Pat. No. 5,800,996; Lee, U.S. Pat. No. 5,945,526; Mathies, U.S. Pat. No. 5,654,419; Lee (1997) Nucleic Acids Res. 25:2816-22). Alternatively, the donor dye and the acceptor dye may be labelled at different attachment sites on the heteroconfigurational oligonucleotide. For example, the heteroconfigurational oligonucleotide may be labelled with a donor dye at the 5′ terminus and an acceptor dye at the 3′ terminus.

Donor and acceptor dyes comprising the energy-transfer dye pair may be any fluorescent moiety which undergoes the energy transfer process, including fluorescein, rhodol, rhodamine, cyanine, phthalocyanine, squaraine, bodipy, coumarin, or benzophenoxazine.

Generally the linker between the donor dye and acceptor dye comprises a structure shown immediately below:

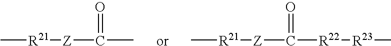

wherein Z is NH, S and O; R²¹ is a C₁-C₁₂ alkyl attached to the donor dye; R²² is a bond, a C₁-C₁₂ alkyldiyl, or a five and six membered ring having at least one unsaturated bond or a fused ring structure which is attached to the carbonyl carbon; and R²³ includes a functional group which attaches the linker to the acceptor dye. R²² may be cyclopentene, cyclohexene, furan, thiofuran, pyrrole, pyrazole, benzene, pyridine, pyrimidine, pyrazine, oxazole, indene, benzofuran, thionaphthene, indole and naphthalene, or substituted forms thereof. Specifically, the linker may have the structure:

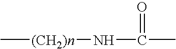

where n ranges from 2 to 10. Generally also, R²³ may comprise the structure:

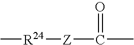

wherein R²⁴ is a C₁-C₁₂ alkyl and Z is as above.

In one embodiment, the linker between the donor dye and acceptor dye includes a functional group which gives the linker some degree of structural rigidity, such as an alkene, diene, an alkyne, a five and six membered ring having at least one unsaturated bond or a fused ring structure. The donor dye and the acceptor dye of the energy-transfer pair may be attached by linkers which comprise the exemplary structures:

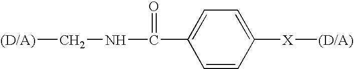

where (D/A) is either a donor dye or an acceptor dye and X may be:

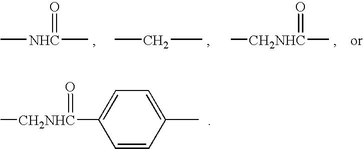

The phenyl rings may be substituted with groups such as sulfonate, phosphonate, and/or other charged groups.

In some embodiments, a heteroconfigurational oligonucleotide or a labelled heteroconfigurational oligonucleotide may be covalently attached by a bond or a linker to a solid-support. Attachment or immobilization of the oligonucleotide may occur: (1) during the synthesis of the oligonucleotide (in situ), or (2) the oligonucleotide may be pre-synthesized, then attached while in solution by a coupling, spotting, immobilizing or deposition process to the solid support.

For example, the solid support may be polystyrene, controlled-pore-glass, silica gel, silica, polyacrylamide, magnetic beads, polyacrylate, hydroxyethylmethacrylate, polyamide, polyethylene, polyethyleneoxy, or copolymers or grafts thereof. In some embodiments, the solid support may comprise small particles, beads, a membrane, a frit, a slide, a plate, a micromachined chip, an alkanethiol-gold layer, a non-porous surface, an addressable array, a gel, or a polynucleotide-immobilizing medium.

In some embodiments, the heteroconfigurational oligonucleotide may be attached to the solid support by a cleavable or non-cleavable linker. Cleavable linkers may be cleaved by chemical reagents, light, or other conditions. For example, a linker may comprise one or more of the following structures:

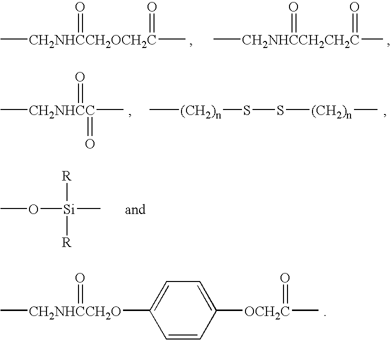

Ester-containing linkers may be cleaved by basic reagents such as aqueous, vaporous, or gaseous ammonium hydroxide (Kempe, U.S. Pat. No. 5,514,789), anhydrous amines (Kempe, U.S. Pat. No. 5,750,672), aqueous hydroxide reagents, and aqueous amines. Ester linkers may be selected on the basis of their cleavage rate and desired stability of the linkage between the quencher moiety and the solid support. For example, an oxalate linkage is relatively labile, being virtually completely cleaved within a few minutes in concentrated ammonium hydroxide at room temperature. A succinate linkage may require one hour or more under the same conditions. Quinone and diglycolate linkages have intermediate stability to basic cleavage. Alkoxysilyl linkers may be cleaved by strong base or fluoride reagents. Disulfide linkers may be cleaved by reducing agents such as dithiothreitol (DTT).

In some embodiments, heteroconfigurational oligonucleotides are synthesized on a solid support using a non-cleavable linker. The oligonucleotide may then be used directly for hybridization or other purposes. Non-cleavable linkers are stable to the acidic, basic, and oxidizing conditions of the phosphoramidite synthesis method. Non-cleavable linkers may include ethyleneoxy units, alkyldiyl, phosphate, and/or amide functionalities.

Heteroconfigurational oligonucleotides, whether labeled or not labeled, may contain various modifications and analogs of standard nucleobases, sugars, and internucleotide linkages. Such modifications and analogs may be disposed at any location and at any appropriate frequency of occurrence in the sequence of the oligonucleotide. Such modifications and analogs may reside in L-form nucleotides, D-form nucleotides, or both.

In addition to the naturally occurring phosphodiester linkages, oligonucleotides of the invention may contain one or more internucleotide linkages comprising a phosphate analog such as a phosphorothioate, a phosphorodithioate, a phosphotriester, or a phosphoramidate. Other internucleotide linkages include those where the sugar/phosphate backbone of DNA or RNA has been replaced with one or more acyclic, achiral, and/or neutral polyamide linkages. One class of internucleotide analogs is the family of peptide nucleic acids (PNAs). The 2-aminoethylglycine polyamide linkage with nucleobases attached to the linkage through an amide bond has been well-studied as an embodiment of PNA and shown to possess exceptional hybridization specificity and affinity (Buchardt, WO 92/20702; Nielsen (1991) Science 254:1497-1500; Egholm (1993) Nature, 365:566-68). PNA can hybridize to its target complement in either a parallel or anti-parallel orientation. However, the anti-parallel duplex (where the carboxyl terminus of PNA is aligned with the 5′ terminus of DNA, and the amino terminus of PNA is aligned with the 3′ terminus of DNA) is typically more stable (Egholm (1993) Nature 365:566-68). PNA probes are known to bind to target DNA sequences with high specificity and affinity (Coull, U.S. Pat. No. 6,110,676). The heteroconfigurational oligonucleotides of the invention include PNA-DNA chimera with discrete PNA and L-form nucleotide sequence portions. They can be synthesized by covalently linking PNA monomers and phosphoramidite nucleosides in virtually any combination or sequence. Efficient and automated methods have been developed for synthesizing PNA-DNA chimera (Vinayak (1997) Nucleosides & Nucleotides 16:1653-56; Uhlmann (1996) Angew. Chem., Intl. Ed. Eng. 35:2632-35; Uhlmann, EP 829542; Van der Laan (1997) Tetrahedron Lett. 38:2249-52; Van der Laan (1998) Bioorg. Med. Chem. Lett. 8:663-68.

Specific examples of nucleobase analogs include, for example, 2,6-diaminopurine, hypoxanthine, pseudouridine, C-5-propyne, isocytosine, isoguanine, or 2-thiopyrimidine.

Sugar modifications at the 2′ or 3′ position include, for example, C₁-C₆ alkoxy, C₁-C₆ alkyl, C₅-C₁₄ aryloxy, C₅-C₁₄ aryl, amino, C₁-C₆ alkylamino, fluoro, chloro, or bromo. Other sugar modifications may include, for example, a 4′-α-anomeric nucleotide, a 1′-α-anomeric nucleotide, a 2′-4′ L-form LNA, a 2′-4′ D-form LNA, a 3′-4′ L-form LNA, or a 3′-4′ D-form LNA. Any of these modifications may occur in an L-form sequence portion, a D-form sequence portion, or both.

Exemplary Synthesis Methods

Heteroconfigurational oligonucleotides can be synthesized on solid supports by the phosphoramidite method (Caruthers, U.S. Pat. No. 4,973,679; Beaucage (1992) Tetrahedron 48:2223-2311), using commercially available phosphoramidite nucleosides (ChemGenes Corp., Ashland, Mass.; Applied Biosystems, Foster City, Calif.) Caruthers, U.S. Pat. No. 4,415,732), supports, e.g. silica, controlled-pore-glass (Caruthers, U.S. Pat. No. 4,458,066) and polystyrene (Andrus, U.S. Pat. Nos. 5,047,524 and 5,262,530) and automated synthesizers such as Models 392, 394, 3948, 3900 and Expedite DNA/RNA Synthesizers (Applied Biosystems, Foster City, Calif.). Oligonucleotide synthesis can be conducted in the common 3′ to 5′ direction of synthesis method with 5′-protected, 3′-phosphorarmidite nucleosides, e.g. IV. Alternatively, oligonucleotide synthesis can be conducted in the 5′ to 3′ direction with 3′-protected, 5′ phosphoramidite nucleosides, e.g. V (Wagner, (1997) Nucleosides & Nucleotides 16:1657-60).

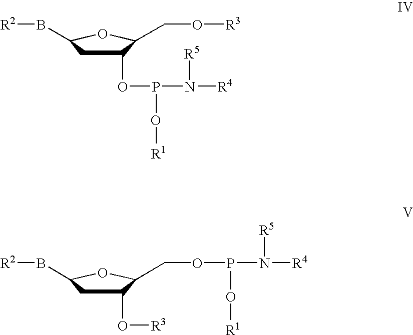

For structures IV and V, exemplary substituents include: wherein R¹ is selected from C₁-C₆ alkyl, substituted C₁-C₆ alkyl (e.g., cyanoethyl), C₅-C₁₄ aryl, and C₅-C₁₄ substituted aryl; R² is an exocyclic nitrogen protecting group such as benzoyl, isobutyryl, acetyl, phenoxyacetyl, aryloxyacetyl, dimethylformamidine, dialkylformamidine, and/or dialkylacetamidine; R³ is an acid-labile protecting group such as DMT, MMT, pixyl, trityl, and trialkylsilyl where alkyl is C₁-C₆; and R⁴ and R⁵ are individually selected from C₁-C₆ alkyl (e.g., isopropyl), substituted C₁-C₆ alkyl, C₅-C₁₄ aryl, and C₅-C₁₄ substituted aryl; or taken together, R⁴ and R⁵ are C₅-C₁₄ cycloalkyl or C₅-C₁₄ heterocycloalkyl.

Exemplary phosphoramidite nucleosides IV and V are the L-form configuration monomers that are typically used for DNA synthesis. Other monomer reagents for preparing the compositions of the present invention include D-form phosphoramidite nucleosides, RNA phosphoramidite nucleosides, 2-aminoethylglycine, and others, with suitable protecting groups. An automated synthesizer may be programmed to deliver any L-form and D-form phosphoramidite nucleoside which is installed on the synthesizer in a reagent delivery bottle during any cycle. Thus, heteroconfigurational oligonucleotides may be synthesized with any sequence of L-form and D-form nucleotides.

L-form and D-form phosphoramidite nucleosides may be prepared and used in oligonucleotide synthesis according to known procedures and methods of sugar and nucleobase protection and phosphitylation of the respective nucleosides. D-form nucleosides are derived from naturally occurring D-DNA sources. L-form phosphoramidite nucleosides may be prepared by any suitable synthetic method. For example, L-form phosphoramidite nucleosides can be prepared from L-ribose, which may be derived from L-xylose in a series of steps (Chu, U.S. Pat. No. 5,753,789; Fujimori (1992) Nucleosides & Nucleotides 11:341-49; Beigelman, U.S. Pat. No. 6,251,666; Furste, WO 98/08856).

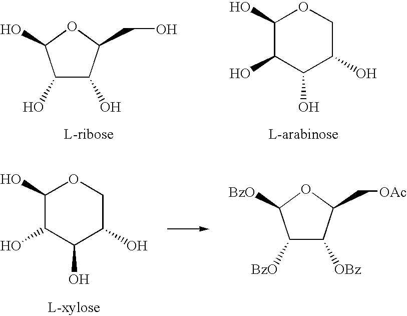

In some embodiments, labelled heteroconfigurational oligonucleotides are synthesized by a method initiated with a labelled solid-support having structure VI:

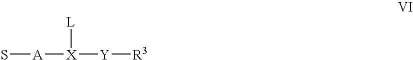

where S is a solid-support; A is a linker; X is a linker with three or more attachment sites; L is a label; Y is selected from O, NH, NR, and S, where R is selected from C₁-C₆ alkyl, C₁-C₆ substituted alkyl, C₅-C₁₄ aryl, and C₅-C₁₄ substituted aryl; and R³ is an acid-cleavable protecting group or a nucleoside having an acid-cleavable protecting group. The labelled solid-support is reacted with an acid reagent to remove the acid-cleavable protecting group. A phosphoramidite nucleoside monomer having an acid-cleavable protecting group R³, and an activator is added to the deprotected labelled solid-support, thereby forming a bond between Y and the 3′ or the 5′ terminus of the nucleoside monomer, which may be an L-form nucleoside or a D-form nucleoside. The solid-support is then treated with an oxidizing reagent to convert the trivalent internucleotide phosphite to phosphate. The steps of: (1) deprotecting the acid-cleavable protecting group, (2) coupling a nucleoside monomer, and (3) oxidation are repeated in a cyclical fashion until the desired sequence of L-form and D-form nucleotides is complete. An additional capping step may be implemented before or after the oxidation step to remove any unreacted 3′ or 5′ hydroxyl groups on the growing oligonucleotide.

In some embodiments, a phosphoramidite label reagent is coupled to a terminus of an oligonucleotide as the final coupling step, thereby labelling the 3′ or 5′ terminus.

Examplary embodiments of labelled solid-support VI include:

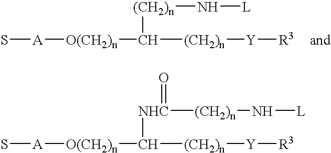

where n is 1 to 12, S is the solid support, and A, L, Y, and R3 are as described above for structure VI.

Another exemplary embodiment of a labelled solid support VI is:

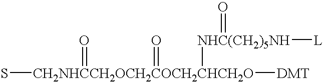

where DMT is 4,4′-dimethoxytrityl.

Another exemplary embodiment of a labelled solid support VI is:

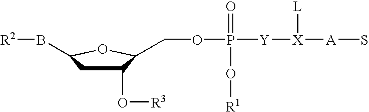

where R¹ is C₁-C₆ alkyl, substituted C₁-C₆ alkyl, C₅-C₁₄ aryl, or C₅-C₁₄ substituted aryl; and R² is an exocyclic nitrogen protecting group such as benzoyl, isobutyryl, acetyl, phenoxyacetyl, aryloxyacetyl, dimethylformamidine, dialkylformamidine, and dialkyl-acetamidine.

For some applications, it may be desirable to prepare a plurality of hetero-configurational oligonucleotides with a common, or conserved, sequence portion, in addition to a unique sequence portion. For example, where a set of heteroconfigurational oligonucleotides are desired with a common L-form nucleotide sequence at the 5′ end and different D-form nucleotide sequences at the 3′ end, synthesis may be initiated with L-form 3′-protected (e.g. DMT), 5′ phosphoramidite nucleosides, e.g. V, on a solid support, in the 5′ to 3′ direction. The solid support will typically be located in a column, tip, well, spot, or other container or location. The synthesis scale can range from a few nanomoles to one or more micromoles, although a larger or smaller scale can also be used. A sequence of L-form nucleotides (e.g., comprising 5 to 50 or more nucleotides) bound to the solid support may be synthesized by the sequential addition of L-form 3′-protected, 5′ phosphoramidite nucleosides. The solid support may be stored for later use, or used immediately. It may be apportioned into a plurality of containers or locations for the subsequent syntheses of different D-form nucleotide sequences. When the solid support is in the form of a bead or particle, a column, tip, or other container may be disassembled and the beads distributed in equal or unequal amounts to two or more columns, tips or other containers and reassembled for sequential addition of D-form 3′-protected, 5′ phosphoramidite nucleosides. When the solid support is a solid surface, membrane, or frit, the support may be divided, crushed, torn, cut, or otherwise apportioned for subsequent and separate syntheses of the D-form nucleotide sequences. The D-form sequence portion syntheses may be conducted in parallel or in series; immediately following the L-form sequence portion synthesis or deferred until the need arises. More generally, D-form and L-form sequence portions can be synthesized separately and later joined together as block polymers, or alternatively, one portion can be synthesized first, followed by sequential addition of monomers having the opposite configuration.

Labelled heteroconfigurational oligonucleotides may be formed by coupling a reactive linking group on a label, e.g. a quencher moiety, with the heteroconfigurational oligonucleotide in a suitable solvent in which both are soluble or appreciably soluble, using methods well-known in the art. For labelling methodology, see Hermanson, Bioconjugate Techniques, (1996) Academic Press, San Diego, Calif. pp. 40-55, 643-71; Garman, 1997, Non-Radioactive Labelling: A Practical Approach, Academic Press, London. Crude, labelled heteroconfigurational oligonucleotides may be purified away from any starting materials or unwanted by-products, and stored dry or in solution for later use, preferably at low temperature.

The label may bear a reactive linking group at one of the substituent positions, e.g. an aryl-carboxyl group of a quencher, or the 5- or 6-carboxyl of fluorescein or rhodamine, for covalent attachment through a linkage. In some embodiments, the linkage that links a label to a heteroconfigurational oligonucleotide should not (i) interfere with hybridization affinity or specificity, (ii) diminish quenching, (iii) interfere with primer extension, (iv) inhibit polymerase activity, or (v) adversely affect the fluorescence, quenching, capture, or hybridization-stabilizing properties of the label. Electrophilic reactive linking groups form a covalent bond with nucleophilic groups such as amines and thiols on a polynucleotide. Examples of electrophilic reactive linking groups include active esters, isothiocyanate, sulfonyl chloride, sulfonate ester, silyl halide, 2,6-dichlorotriazinyl, phosphoramidite, maleimide, haloacetyl, epoxide, alkylhalide, allyl halide, aldehyde, ketone, acylazide, anhydride, and iodoacetamide. Active esters include succinimidyl (NHS), hydroxybenzotriazolyl (HOBt) and pentafluorophenyl esters.

An NHS ester of a label reagent may be preformed, isolated, purified, and/or characterized, or it may be formed in situ and reacted with a nucleophilic group of a heteroconfigurational oligonucleotide. Typically, a label carboxyl group is activated by reacting with a combination of: (1) a carbodiimide reagent, e.g. dicyclohexylcarbodiimide, diisopropylcarbodiimide, EDC (1-ethyl-3-(3-dimethylaminopropyl)carbodiimide); or a uronium reagent, e.g. TSTU (O—(N-Succinimidyl)-N,N,N′,N′-tetramethyluronium tetrafluoroborate, HBTU (O-benzotriazol-1-yl)-N,N,N′,N′-tetramethyluronium hexafluorophosphate), or HATU (O-(7-azabenzotriazol-1-yl)-N,N,N′,N′-tetramethyluronium hexafluorophosphate); and (2) an activator, such as HOBt (1-hydroxybenzotriazole) or HOAt (1-hydroxy-7-azabenzotriazole; and (3) N-hydroxysuccinimide to give the NHS ester.

An exemplary non-nucleosidic phosphoramidite label reagent has the general formula VII:

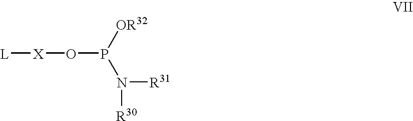

where L is a protected or unprotected form a label; X is a linker or bond; R³⁰ and R³¹ taken separately are C₁-C₁₂ alkyl, C₄-C₁₀ aryl, and/or cycloalkyl containing up to 10 carbon atoms, or R³⁰ and R³¹ taken together with the phosphoramidite nitrogen atom form a saturated nitrogen heterocycle; and R³² is a phosphite ester protecting group which prevents extension of the oligonucleotide (Theisen (1992) “Fluorescent dye phosphoramidite labelling of oligonucleotides”, in Nucleic Acid Symposium Series No. 27, Oxford University Press, Oxford, pp. 99-100). Generally, R³² is stable to oligonucleotide synthesis conditions and is able to be removed from a synthetic oligonucleotide product with a reagent that does not adversely affect the integrity of the heteroconfigurational oligonucleotide or the label. Exemplary R³² substituents include (i) methyl, (ii) 2-cyanoethyl; —CH₂CH₂CN, or (iii) 2-(4-nitrophenyl)ethyl; and CH₂CH₂(p-NO₂Ph). Exemplary embodiments of phosphoramidite label reagents include those wherein: (i) R³⁰ and R³¹ are each isopropyl, (ii) R³⁰ and R³¹ taken together is morpholino, (iii) X is C₁-C₁₂ alkyl, and (iv) R³² is 2-cyanoethyl. Alternatively, linker X, may be:

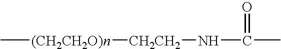

where n ranges from 1 to 10. An exemplary phosphoramidite labelling reagent has structure VIII:

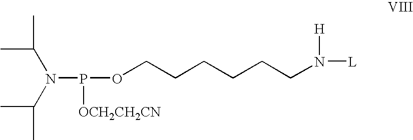

A phosphoramidite label reagent VII or VIII reacts with a hydroxyl group, e.g. 5′ terminal OH of a heteroconfigurational oligonucleotide covalently attached to a solid support, under mild acid activation, e.g. tetrazole, to form an internucleotide phosphite group which is then oxidized to an internucleotide phosphate group. In some instances, the phosphoramidite label reagent contains functional groups that require protection either during the synthesis of the reagent or during its subsequent use to label a heteroconfigurational oligonucleotide. The protecting group(s) used will depend upon the nature of the functional groups, and will be apparent to those having skill in the art (Greene, T. and Wuts, P. Protective Groups in Organic Synthesis, 2nd Ed., John Wiley & Sons, New York, 1991). The label will be attached at the 5′ terminus of the oligonucleotide, as a consequence of the common 3′ to 5′ direction of synthesis method with 5′-protected, 3′-phosphoramidite nucleosides, e.g. IV. Alternatively, the 3′ terminus of an oligonucleotide may be labelled with a phosphoramidite label reagent when synthesis is conducted in the 5′ to 3′ direction with 3′-protected, 5′ phosphoramidite nucleosides, e.g. V (Vinayak, U.S. Pat. No. 6,255,476).

Other phosphoramidite label reagents, both nucleosidic and non-nucleosidic, allow for labelling at other sites of a heteroconfigurational oligonucleotide, e.g. 3′ terminus, nucleobase, internucleotide linkage, sugar. Labelling at the nucleobase, internucleotide linkage, and sugar sites allows for internal and multiple labelling.

L-Form Oligonucleotide Arrays

In some embodiments, the present invention includes an array of immobilized L-form nucleotide-containing oligonucleotides. The L-form nucleotide-containing oligonucleotides (also referred to herein as “L-form polynucleotides” or “L-form oligonucleotides”) comprise a sequence of L-form nucleotides that is capable of hybridizing to its L-form complement in a target polynucleotide (e.g., to an L-form sequence portion of a heteroconfigurational oligonucleotide). Typically, the L-form sequence portion will be at least five L-nucleotides in length, and may be as many as 100 or more. The array can comprise two to many thousands of unique or identical sequences of L-form nucleotide-containing oligonucleotides. In one embodiment, each location on the array will have a pre-selected quantity of a unique sequence, e.g. 1 picomole to 1 nanomole.

In some embodiments, immobilized oligonucleotides comprise heteroconfigurational oligonucleotides of the invention. In some embodiments, immobilized oligonucleotides do not comprise heteroconfigurational oligonucleotides. In some embodiments, immobilized oligonucleotides contain L-form nucleotides but not D-form nucleotides.

In an array of the present invention, one or more L-form oligonucleotides is immobilized at each addressable location. The addressable locations may be an arrangement of vessels, segregated areas, spots, or other configurations such that reagents, light, heating, cooling, or other operations can be deliberately directed to discrete locations. The array may provide for operations common to all locations, such as washing each location in parallel by flooding an array surface, or directing light to the entire surface, or applying vacuum pressure to each well of a multi-well microtiter plate.

In some embodiments, the supports in the arrays may comprise one or more membrane, beads, or coated or uncoated particles. Supports may comprise a magnetic or paramagnetic material.

Supports may comprise bound or immobilized spatially addressable L-form nucleotide oligonucleotides that comprise pre-determined capture sequence(s), or specific ligands.

The arrays and supports of the present invention may have a wide variety of geometries and configurations, and be fabricated using any one of a number of different known fabrication techniques. Exemplary fabrication techniques include, but are not limited to, in situ synthesis techniques (Southern, U.S. Pat. No. 5,436,327); light-directed in situ synthesis techniques, (Fodor, U.S. Pat. No. 5,744,305); robotic spotting techniques (Cheung, (1999) Nature Genetics, 21: 15-19; Brown, U.S. Pat. No. 5,807,522; Cantor, U.S. Pat. No. 5,631,134; Drmanac, U.S. Pat. No. 6,025,136); or arrays of beads having oligonucleotides attached thereto (Walt, U.S. Pat. No. 6,023,540). The solid support of the invention also includes a plurality of L-form oligonucleotides immobilized on silicon wafers disposed in microtiter plates (Rava, U.S. Pat. No. 5,545,531). Furthermore, the present invention also includes a plurality of L-form oligonucleotides immobilized on microspheres or beads which are affixed, settled, or otherwise disposed on the terminal end of an optical fiber. Array compositions may be fabricated from bundles of optical fibers. Detectable signals from labelled L-form oligonucleotides or their labelled hybridization complexes can generate unique optical signatures which are decoded to correlate the location of an individual location with the hybridizing sequence (Walt, U.S. Pat. Nos. 5,244,636 and 5,250,264).

One embodiment of an immobilized L-form nucleotide-containing oligonucleotide has structure IX:

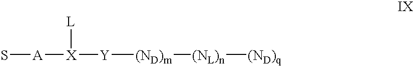

where S, A, X, and Y are as described for structure VI above. NL is a sequence of L-form nucleotides; ND is a sequence of D-form nucleotides; m is an integer from 0 to 100; n is an integer from 5 to 100; and q is an integer from 0 to 100. In some embodiments, q=0 and m>0. In some embodiments, m=0.

In some embodiments, the immobilized L-form nucleotide-containing oligonucleotide contains at least 5 L-form nucleotides, and may or may not contain D-form nucleotides. Any D-form nucleotide in the oligonucleotide may appear at any part of the sequence. Therefore, structure IX may also have the following embodiments:

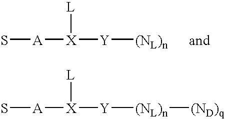

as well as embodiments that have more sequence portions of L-form and D-form nucleotides.

The solid support may comprise any suitable material, such as polystyrene, a glass such as controlled-pore-glass, silica gel, silica, polyacrylamide, magnetic beads, polyacrylate, hydroxyethylmethacrylate, polyamide, polyethylene, polyethyleneoxy, and/or copolymers or grafts thereof. The form of the solid support may be small particles, beads, membranes, frits, slides, plates, micromachined chips, alkanethiol-gold layers, non-porous surfaces, addressable arrays, or polynucleotide-immobilizing media. In one embodiment, the solid support comprises a nylon membrane. In another embodiment, the solid support comprises a polystyrene bead.

Exemplary Hybridization Methods

The present invention includes methods of forming polynucleotide hybrids, by providing a heteroconfigurational polynucleotide comprising a D-form polynucleotide sequence portion and an L-form polynucleotide sequence portion that is covalently linked to the D-form polynucleotide sequence portion, and hybridizing the heteroconfigurational polynucleotide to at least a first complementary polynucleotide to form a duplex between the first complementary polynucleotide and (1) the L-form polynucleotide sequence portion, (2) the D-form polynucleotide sequence portion, or both (1) and (2).

In some embodiments, a hybrid is formed by hybridizing a heteroconfigurational polynucleotide to a first complementary polynucleotide that is complementary to all or part of the L-form polynucleotide sequence portion. In some embodiments, a hybrid is formed by hybridizing a heteroconfigurational polynucleotide to a first complementary polynucleotide that is complementary to all or part of the D-form polynucleotide sequence portion. In some embodiments, a hybrid is formed between a heteroconfigurational polynucleotide, a first complementary polynucleotide that is complementary to all or part of the D-form polynucleotide sequence portion, and a second complementary polynucleotide that is complementary to all or part of the L-form polynucleotide sequence portion. In some embodiments such as described above, hybridization is performed in solution, when neither the heteroconfigurational polynucleotide nor the complementary polynucleotide(s) are attached or immobilized on a solid support.

In some embodiments, a hybrid comprising a heteroconfigurational polynucleotide is captured or immobilized on a solid support. In some embodiments, the hybrid comprises a heteroconfigurational polynucleotide and a first complementary polynucleotide that is hybridized to all or part of the L-form polynucleotide sequence portion, wherein the first complementary polynucleotide is attached to a solid support. In some embodiments, the hybrid comprises a heteroconfigurational polynucleotide and a first complementary polynucleotide that is hybridized to all or part of the L-form polynucleotide sequence portion, wherein the heteroconfigurational polynucleotide is attached to a solid support. In some embodiments, the hybrid comprises a heteroconfigurational polynucleotide and a first complementary polynucleotide that is hybridized to all or part of the D-form polynucleotide sequence portion, wherein the first complementary polynucleotide is attached to a solid support. In some embodiments, the hybrid comprises a heteroconfigurational polynucleotide and a first complementary polynucleotide that is hybridized to all or part of the D-form polynucleotide sequence portion, wherein the heteroconfigurational polynucleotide is attached to a solid support. In some embodiments, a hybrid is formed between a heteroconfigurational polynucleotide, a first complementary polynucleotide that is complementary (and hybridized) to all or part of the D-form polynucleotide sequence portion, and a second complementary polynucleotide that is complementary to (and hybridized to) all or part of the L-form polynucleotide sequence portion, wherein the first complementary polynucleotide or the second complementary polynucleotide or the heteroconfigurational polynucleotide is attached to a solid support. In the embodiments above, attachment or immobilization can be accomplished covalently or non-covalently. Also, in the embodiments above, hybrids can be formed either before, during, or after immobilization, attachment, or capture on a support.

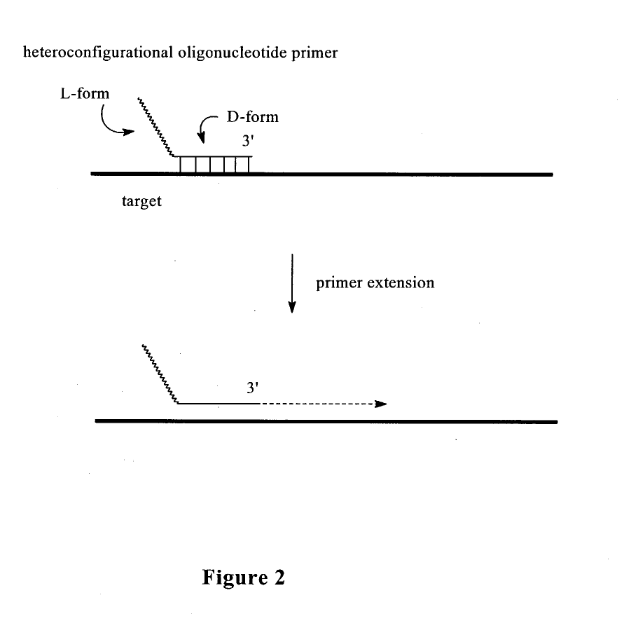

The hybrid may comprise one or more duplex, triplex, or other high-ordered structures where at least the nucleobases of the L-form sequence portion or the D-form sequence portion of the heteroconfigurational oligonucleotide pair with corresponding nucleobases in a complementary polynucleotide by specific interactions. In some embodiments, the heteroconfigurational oligonucleotide includes an L-form sequence portion having 5 to 50 L-nucleotides covalently attached by a bond or a linker to a D-form sequence portion having 5 to 50 D-nucleotides. FIG. 2 shows hybridization of an exemplary heteroconfigurational oligonucleotide (upper structure) with a complementary “target” polynucleotide (lower structure). In this illustrative embodiment, the D-form sequence portion of the heteroconfigurational oligonucleotide hybridizes to all or part of a D-form complement in the target.

Methods to perform the hybridization with the oligonucleotides of the invention will vary depending upon the nature of the support-bound capture polynucleotide and the polynucleotide in solution that is to be captured (Bowtell, (1999) Nature Genetics, 21: 25-32; Brown, (1999) Nature Genetics, 21: 33-37). Additional references for hybridization can be found in WO 02/02823 A2 and references cited therein.

In some embodiments, either or both of the heteroconfigurational oligonucleotide and the target polynucleotide (or complementary oligonucleotide) is/are covalently attached to one or more labels. Labels may produce a detectable signal, or facilitate a detectable signal by subsequent reaction, conversion, or interaction with other reagents. Alternatively or additionally, labels may stabilize hybridization, promote primer extension, or enable capture, complexation, or sequestration of the labelled heteroconfigurational oligonucleotide/target hybrid or products derived therefrom. In some embodiments, the label may be a fluorescent dye, a quencher, an energy-transfer dye, a quantum dot, digoxigenin, biotin, a mobility-modifier, a polypeptide, a hybridization-stabilizing moiety, and a chemiluminescent precursor.

A hybrid comprising a heteroconfigurational oligonucleotide and one or more complementary oligonucleotides may be formed by hybridization in a mixture containing a plurality of target polynucleotides having different sequences. Nonhybridized target polynucleotides may then be separated from the hybrid if desired, and the hybrid may be detected. In some embodiments, such a separation step is unnecessary because the hybrid can be detected in a homogeneous format, wherein a detectable signal is created by hybridization between the heteroconfigurational oligonucleotide and a complementary target.

In some embodiments, the target polynucleotide comprises an SNP-containing nucleic acid, an mRNA, a cRNA, a cDNA, or genomic DNA. In some embodiments, the target comprises a synthetic polynucleotide sequence or sequence portion that is complementary to the heteroconfigurational oligonucleotide.

A hybridized heteroconfigurational oligonucleotide may include a reporter and a quencher. The reporter or the quencher may be each covalently attached by a bond or a linker to the L-form sequence portion or the D-form sequence portion of the heteroconfigurational oligonucleotide. For example, the reporter may be attached by a linker to the L-form sequence portion and the quencher may be attached by a linker to the D-form sequence portion.

In some embodiments, hybridization may be conducted while the target polynucleotide is immobilized on a solid support.

A labelled heteroconfigurational oligonucleotide/target hybrid may be denatured and the labelled heteroconfigurational oligonucleotide then hybridized to another oligonucleotide which has a complementary L-form sequence portion to form a heteroconfigurational oligonucleotide/L-polynucleotide hybrid. Configurational specificity is an advantageous property of heteroconfigurational oligonucleotides, where their L-form sequence portion only hybridizes to a complementary L-form sequence portion and likewise, where their D-form sequence portion only hybridizes to a complementary D-form sequence portion. This configuration specificity, i.e. orthogonality, minimizes or eliminates cross-hybridization between the targetting step and the capture step, common to many nucleic acid hybridization assays.

While L-form and D-form polynucleotide sequences do not base pair with each other in a stable manner, their properties in an achiral environment are necessarily equivalent. For example, synthesis efficiencies of the mirror image phosphoramidite nucleosides by the phosphoramidite method of synthesis must be equivalent. Chemical labelling reactions with achiral labelling reagents are equally efficient. Purification and analysis can be conducted by the same methods and give the same results for the mirror image, enantiomeric L-form and D-form oligonucleotides, as long as the environments are achiral. For example, typical reverse-phase HPLC analysis will give the same profile and retention time for mirror image L-form and D-form oligonucleotides. It should be noted however, that identical sequence heteroconfigurational oligonucleotides where the individual nucleotides are not of the same L-form and D-form configurations are diastereomers and do not have the same properties.

The hybridization properties of L-form duplexes are inherently equivalent, although orthoganal, to D-form duplexes. For example, an all L-form oligonucleotide of a particular sequence has the same Tm in binding to its L-form complement oligonucleotide as does an all D-form oligonucleotide of the same sequence in binding to its D-form complement. The presence of a non-complementary L-form or D-form sequence portion in a heteroconfigurational oligonucleotide in a duplex may have some effect on affinity, either stabilizing or destabilizing.

Target sequence-specific portions of the heteroconfigurational oligonucleotide are of sufficient length to permit specific annealing to complementary target sequences. Detailed descriptions of probe design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990), for example.

The fluorescent/quencher heteroconfigurational oligonucleotide probes of the invention are useful as detection agents in a variety of DNA amplification/quantification strategies including, for example, 5′-nuclease assay, Strand Displacement Amplification (SDA), Nucleic Acid Sequence-Based Amplification (NASBA), Rolling Circle Amplification (RCA), Oligonucleotide Ligation Assay (OLA), Ligase Chain Reaction (LCR) (Barany, U.S. Pat. No. 5,494,810), Ligase Detection Reaction (LDR) (Barany, U.S. Pat. Nos. 6,312,892 and 6,027,889), Transcription-Mediated Amplification (TMA) and Q-beta replicase. Fluorescent/quencher heteroconfigurational oligonucleotide probes are also useful for direct detection of targets in other solution phase or solid phase (e.g., array) assays. Furthermore, the probes can be used in any format, including, for example, molecular beacons, Scorpion probes™, Sunrise probes™, light up probes, Invader™ Detection probes, and TaqMan™ probes. See, for example, Cardullo, R. (1988) Proc. Natl. Acad. Sci. USA, 85:8790-8794; Stryer, L., (1978) Ann. Rev. Biochem., 47:819-846; Rehman, F. N., (1999) Nucleic Acids Research, 27:649-655; Gibson, E. M., (1996) Genome Methods, 6:995-1001; Livak, U.S. Pat. No. 5,538,848; Wittwer, C. T., (1997) BioTechniques, 22:176-181; Wittwer, C. T., (1997) BioTechniques, 22:130-38; Tyagi, WO 95/13399, Tyagi, U.S. Pat. Nos. 6,037,130; 6,150,097; and 6,103,476; Uchara, (1999) BioTechniques, 26:552-558; Whitcombe, (1999) Nature Biotechnology, 17:804-807; Lyamichev, (1999) Nature Biotechnology, 17:292; Daubendiek, (1991) Nature Biotechnology, 15:273-350; Nardone, WO 99/64432; Nadeau, U.S. Pat. Nos. 5,846,726 and 5,928,869; and Nazarenko, U.S. Pat. No. 5,866,336.

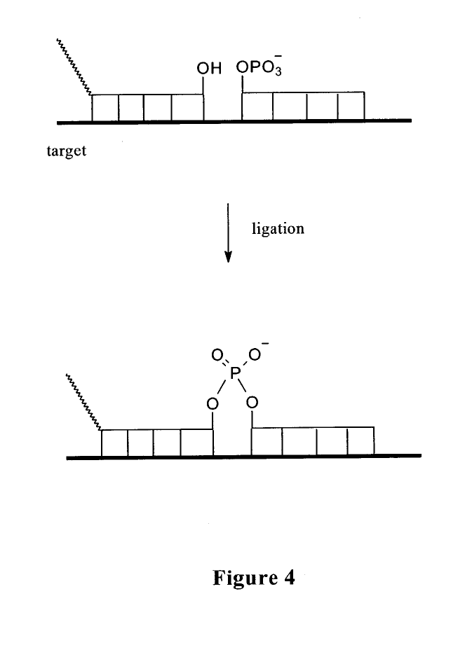

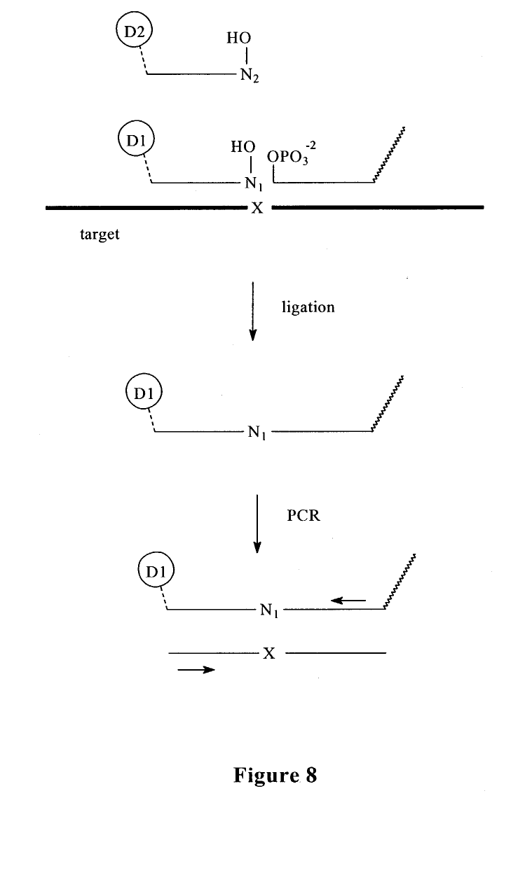

In some embodiments, the present invention includes a method where a labelled heteroconfigurational oligonucleotide probe and a second oligonucleotide probe are adjacently hybridized, as a probe set, to a target polynucleotide. Under appropriate conditions, adjacently hybridized probes may be ligated together to form a ligation product, provided that they comprise appropriate reactive groups, for example, without limitation, a free 3′-hydroxyl or 5′-phosphate group prior to ligation (e.g., see FIG. 4). Some ligation reactions may comprise more than one heteroconfigurational oligonucleotide probe or more than one second probe to allow sequence discrimination between target sequences that differ by one or more nucleotides (FIG. 8).

In some embodiments, a target sequence comprises an upstream or 5′ region, a downstream or 3′ region, and an SNP nucleotide located between the upstream region and the downstream region. The SNP is a nucleotide that is to be detected by a pair of ligatable probes (“probe set”) and may represent, for example, a single polymorphic nucleotide in a multiallelic target locus. In some embodiments, a nucleotide base complementary to an SNP site of the target may be present on the proximal end of either a heteroconfigurational oligonucleotide probe (a first probe) or a second probe of a target-specific probe pair. When the probes of the probe set are hybridized to the appropriate upstream and downstream target regions, and the nucleotide base complementary to the SNP is base-paired with the SNP on the target sequence, the hybridized probes may be ligated together to form a ligation product (FIG. 8). A mismatched base at the nucleotide base complementary to the SNP, however, interferes with ligation, even if both probes are otherwise fully hybridized to their respective target regions. Thus, highly related sequences that differ by as little as a single nucleotide can be distinguished.

FIG. 8 shows an exemplary ligation reaction. Two potential alleles in a biallelic locus can be distinguished by combining a probe set comprising: (1) two fluorescent dye-labelled probes, their sequences differing only in their SNP complementary sites (N₁ and N₂) at their terminii, either 3′ or 5′, (2) a phosphorylated heteroconfigurational oligonucleotide probe, where the wavy line is an L-form sequence portion, and (3) the sample containing the target. The two fluorescent dyes, D1 and D2, are different and spectrally distinct. All three probes will hybridize with the target sequence under appropriate conditions, but only the dye-labelled probe with the hybridized SNP complement, will be ligated with the hybridized phosphorylated heteroconfigurational oligonucleotide probe. The probe with the terminal nucleoside complementary to X (N₁) ligates to the 5′ phosphate-heteroconfigurational oligonucleotide probe and the probe with the mismatch terminal nucleoside (N₂) does not. For example, if only one allele is present in the sample where the SNP site X is a G nucleotide, and N₁ is C and N₂ is T, then only the probe where N₁ is C will ligate to form the ligation product. Where the ligation product can be separated from unligated N₂ probe or detected separately or be detectably distinguished, then detection of label D1 indicates that the SNP site was G. If both labels D1 and D2 can be detected, then it can be inferred that both allelic forms (X G and A) were present from a heterozygous individual.

In some embodiments, probe sets do not comprise an SNP complement locus at the terminus of the first or the second probe. Rather, the target SNP locus nucleotide or nucleotides to be detected are located within either the 5′ or 3′ target region. The nucleotides to be detected may be both terminal or internal. Probes with target-specific portions that are filly complementary with their respective target regions will hybridize under high stringency conditions. Probes with one or more mismatched bases in the target-specific portion, by contrast, will not hybridize to their respective target region. Both the heteroconfigurational oligonucleotide first probe and the second probe must be hybridized to the target for a ligation product to be generated.

In some embodiments, the heteroconfigurational oligonucleotide probes and second probes in a probe set are designed with similar melting temperatures (Tₘ). Where a probe includes an SNP site, the Tₘ for the probe(s) comprising the SNP site complement(s) may be designed to be approximately 4-6° C. lower than the other probe(s) that do not contain the SNP site complement in the probe set. The probe comprising the SNP site complement(s) may also be designed with a Tₘ near the ligation temperature. Thus, a probe with a mismatched nucleotide will more readily dissociate from the target at the ligation temperature. The ligation temperature, therefore, provides another way to discriminate between, for example, multiple potential alleles in the target.

A ligation agent according to the present invention may comprise any number of enzymatic or chemical (i.e., non-enzymatic) agents. For example, ligase is an enzymatic ligation agent that, under appropriate conditions, forms phosphodiester bonds between the 3′-OH and the 5′-phosphate of adjacent nucleotides in DNA or RNA molecules when they are hybridized to a complementary sequence. Temperature sensitive ligases, include, but are not limited to, bacteriophage T4 ligase and E. coli ligase. Thermostable ligases include, but are not limited to, Taq ligase, Tth ligase, and Pfu ligase. Thermostable ligase may be obtained from thermophilic or hyperthermophilic organisms.

Chemical ligation agents for coupling probes include, without limitation, activating, condensing, and reducing agents, such as carbodiimide reagents, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the invention. The internucleotide linkage may be a phosphodiester linkage. Other exemplary internucleotide linkages include disulfide, phosphoramidate, acetal, pyrophosphate, and those formed between appropriate reactive groups such as an α-haloacyl group and a phosphothioate group to form a thiophosphorylacetylamino group, and a phosphorothioate and a tosylate or iodide group to form a phosphorothioester. Detailed protocols for chemical ligation methods and descriptions of appropriate reactive groups can be found, among other places, in Xu, (1999) Nucleic Acid Res., 27:875-81; Gryaznov, (1993) Nucleic Acid Res. 21:1403-08; Gryaznov, (1994) Nucleic Acid Res. 22:2366-69; Kanaya, (1986) Biochemistry 25:7423-30; Luebke, (1992) Nucleic Acids Res. 20:3005-09; Sievers, (1994) Nature 369:221-24; Liu, (1999) Nucleic Acids Res. 26:3300-04; Wang, (1994) Nucleic Acids Res. 22:2326-33; Purmal, (1992) Nucleic Acids Res. 20:3713-19; Ashley, (1991) Biochemistry 30:2927-33; Chu, (1988) Nucleic Acids Res. 16:3671-91; Sokolova, (1988) FEBS Letters 232:153-55; Naylor, (1966) Biochemistry 5:2722-28; and Letsinger, U.S. Pat. No. 5,476,930.

Ligation comprises at least one cycle of ligation. In some embodiments, more than one cycle is performed comprising: (1) hybridizing the target-specific portions of a first probe and a second probe, that are suitable for ligation, to their respective complementary target regions; (2) ligating the 3′ end of the first probe with the 5′ end of the second probe to form a ligation product; and (3) denaturing the nucleic acid duplex to separate the ligation product from the target strand. The cycle may, or may not be, repeated by thermal cycling the ligation reaction to linearly increase the amount of ligation product.

After ligation, the ligation product may be hybridized to a “capture” oligonucleotide. The capture oligonucleotide may be immobilized on a solid support and configured in an addressable array. The L-form nucleotide portion (“tag”) of the ligation product may be complementary to an L-form nucleotide sequence portion of an immobilized oligonucleotide.

Also within the scope of the invention are ligation techniques such as gap-filling ligation, including, without limitation, gap-filling OLA and LCR, bridging oligonucleotide ligation, and correction ligation (e.g., see Ullman, U.S. Pat. No. 5,185,243; Backman, EP 320308; EP 439182, and WO 90/01069).

In some applications, target sequence detection may be impeded due to low target copy number or low detection sensitivity. Target sequences may be amplified using any suitable method such as the polymerase chain reaction (PCR), detailed in M. Innis, PCR Protocols, Academic Press, New York (1990). In some embodiments, after ligation, the ligation product can be amplified by PCR by a specific set of primers (e.g., see F. Barany et al., WO 97/45559).

Optionally, a ligation product may be purified by any process that removes at least some unligated probes, target DNA, enzymes or accessory agents from the ligation reaction mixture following at least one cycle of ligation. Such processes include, but are not limited to, molecular weight/size exclusion processes, e.g., gel filtration chromatography or dialysis, sequence-specific hybridization-based pullout methods, affinity capture techniques, precipitation, electrophoresis, chromatography, adsorption, or other nucleic acid purification techniques. The skilled artisan will appreciate that purifying the ligation product prior to amplification reduces the quantity of primers needed to amplify the ligation product, thus reducing the cost of detecting a target sequence. Also, purifying the ligation product prior to amplification decreases possible side reactions during amplification and reduces competition from unligated probes during hybridization.

In some embodiments, the present invention includes methods comprising primer extension, wherein a heteroconfigurational oligonucleotide primer hybridizes to a target polynucleotide to form a heteroconfigurational oligonucleotide/target hybrid. In some embodiments, the heteroconfigurational oligonucleotide primer includes an L-form sequence portion having 5 to 50 L-nucleotides covalently attached by a bond or a linker to a D-form sequence portion having 5 to 50 D-nucleotides. In some embodiments, the 3′ terminus nucleotide of the D-form sequence portion has a 3′ hydroxyl. The 3′ terminus of the D-form sequence portion of the labelled heteroconfigurational oligonucleotide strand of the hybrid is extended with a primer extension reagent. The bottom structure of FIG. 2 shows primer extension of a heteroconfigurational oligonucleotide/target hybrid where the dotted arrow illustrates incorporation of nucleotide 5′-triphosphates in the synthesis of a nucleic acid strand from the 3′ terminus of the heteroconfigurational oligonucleotide primer of the duplex. The reaction comprises a polymerase, one or more enzymatically-incorporatable nucleotide 5′-triphosphates, and buffer. By the primer extension method, one or more labelled polynucleotide fragments may be formed.

Amplification according to the present invention encompasses a broad range of techniques for amplifying nucleic acid sequences, either linearly or exponentially. Examples of such techniques include, but are not limited to, in vitro transcription, PCR and other methods employing a primer extension step. Amplification methods may comprise thermal-cycling or may be performed isothermally. Amplification methods generally comprise at least one cycle of amplification, i.e., the sequential procedures of: hybridizing primers to primer-specific portions of a ligation product or target sequence; synthesizing a strand of nucleotides in a template-dependent manner using a polymerase; and denaturing the newly-formed nucleic acid duplex (amplicon) to separate the strands. The cycle may or may not be repeated.

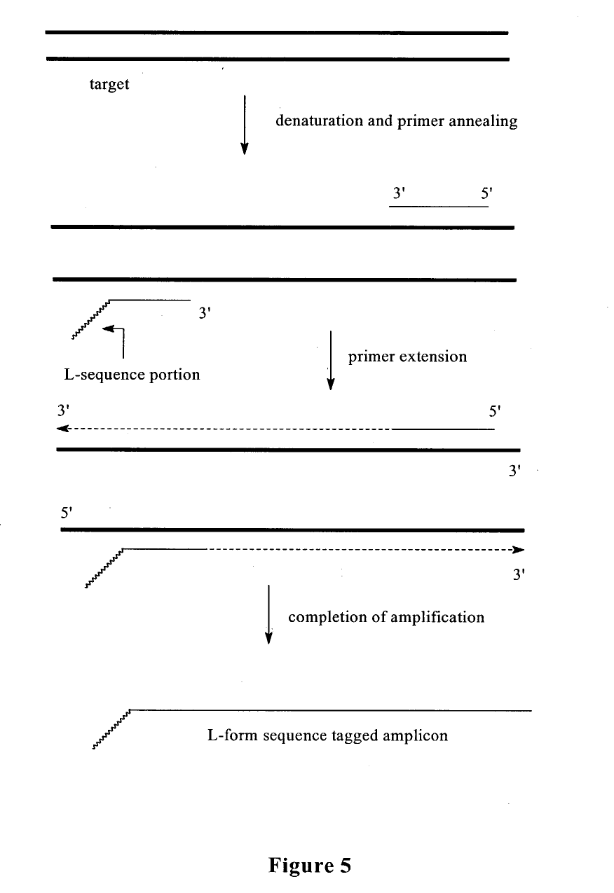

FIG. 5 shows an exemplary polymerase chain reaction using a heteroconfigurational oligonucleotide primer. Primer extension through the 3′ end of a D-form sequence portion of the heteroconfigurational oligonucleotide primer incorporates an L-form sequence portion as a “tag” in the PCR amplicon. Since the L-form nucleotides do not form stable base-pairs with D-form nucleotide, the target portion which is amplified is limited to the D-form nucleotides of the primers. After amplification, the 5′ terminus of one strand of the resulting amplicon comprises an L-form sequence tag.

In some embodiments, methods of the invention comprise methods and assays for monitoring the relative concentrations of mRNA of interest. An mRNA population can be isolated from a sample, e.g. tissue, and converted to the more stable cDNA by reverse transcriptase. One method to copy mRNA or cDNA sequences is to take advantage of the poly-A tail at the 3′ end of mRNA with poly-A and poly-T containing primers. Alternatively, gene specific primers can be used to copy, e.g. amplify, particular cDNA of interest. Methods to copy mRNA and cDNA include PCR, rolling circle amplification, and in vitro transcription (IVT). In some embodiments, mRNA species are detected or quantified using an array comprising a plurality of different sequence specific tags.

Heteroconfigurational oligonucleotide primers are also useful in IVT (in vitro transcription) where the primer sequence includes a T7 RNA polymerase promoter sequence at the 5′ end. Many copies of RNA (cRNA) can be transcribed from each cDNA molecule. For example, labels can be incorporated directly via labelled ribonucleotide 5′-triphosphates, or in a second reverse transcriptase reaction to produce labelled cDNA. Labelled cDNA and cRNA can be hybridized to their complementary sequences immobilized on solid support. In some embodiments, the L-form sequence portion of a cDNA from primer extension of a heteroconfigurational oligonucleotide primer can hybridize to a complementary L-form sequence portion of a complementary oligonucleotide that is immobilized on a support.

Arrays and methods of making them are well known, as described, for example, in WO 02/02823 and references cited therein, and in Microarray Biochip Technology, M. Schena Ed., Eaton Publixhing, BioTechniques Books Division, Natick, Mass. 01760, for example. In some embodiments, a universal L-DNA array is spotted onto a porous membrane mounted to the bottom of a 96 well microtitre plate made from PTFE, hydrophil (Multiscreen Resist-R1, Millipore), polypropylene (AcroWell Plate, Pall), or nylon (Cuno-white, Cuno). For example, in some embodiments, approximately 1-15 nmole of oligonucleotide is immobilized per 4.5 mm diameter well.

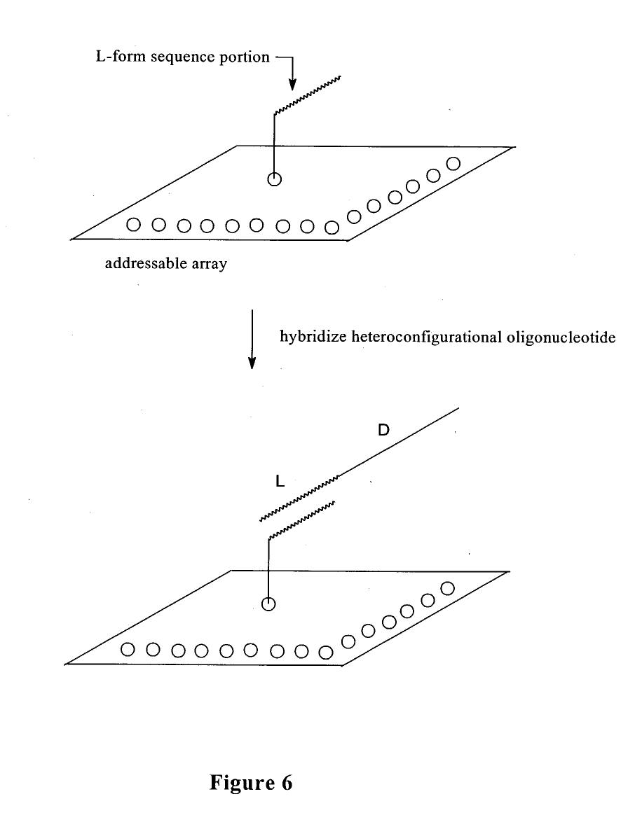

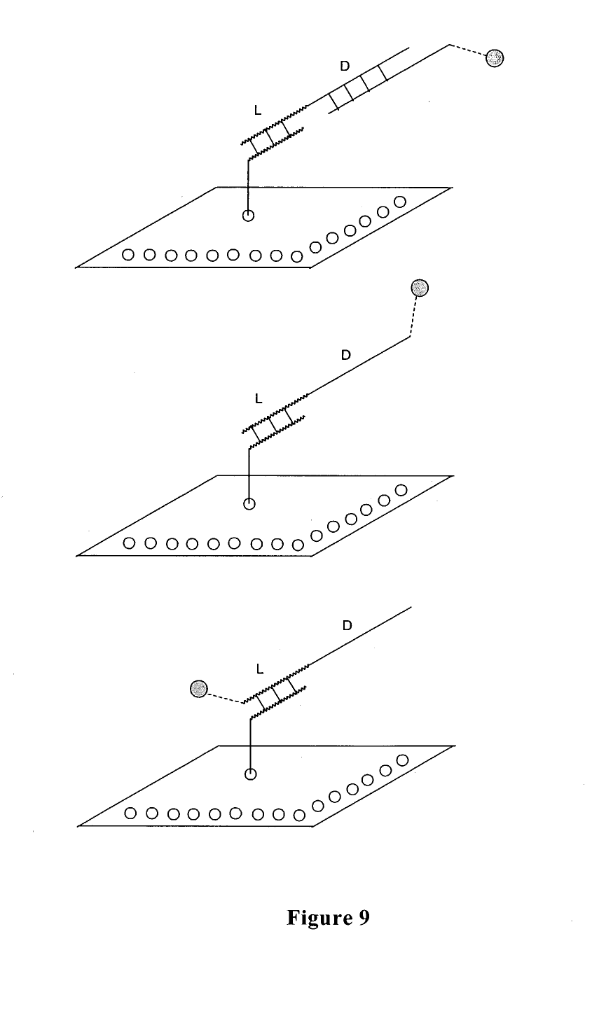

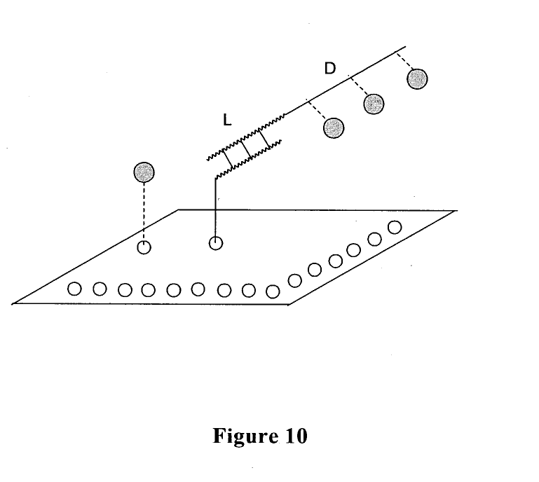

A plurality of immobilized oligonucleotides can be arrayed at addressable locations (FIG. 6). At each location there may be an immobilized oligonucleotide with a different L-form sequence. If the cDNA is labelled, its L-form sequence can be deduced by the presence or absence of detectable signal from any particular loci. A number of different labelling orientations are feasible (FIG. 9). Labelled control positions may establish baseline, background values and provide normalization of signal (FIG. 10).

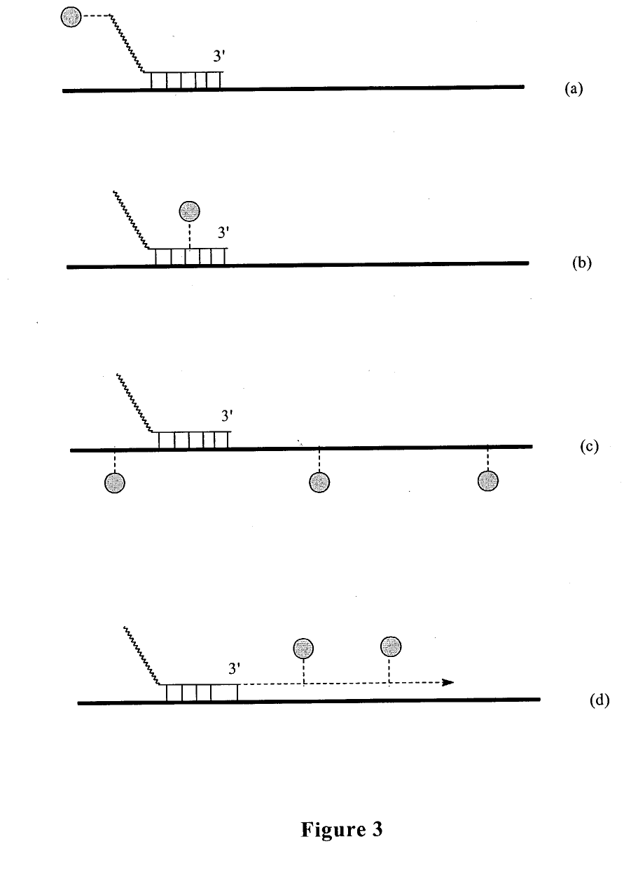

The present invention also includes methods for gene expression analysis where the target polynucleotide is a cDNA and the cDNA is formed by hybridizing a heteroconfigurational oligonucleotide primer to an RNA target polynucleotide to form a primer/target hybrid and extending the 3′ of the primer of the primer/target hybrid with a primer extension reagent to form a cDNA transcript. The primer extension reaction includes at least a reverse transcriptase enzyme, one or more nucleotide 5′-triphosphates, and a buffer. One or more of the nucleotide 5′-triphosphates may be labelled to generate a multiply labelled transcript cDNA, tagged with an L-form DNA portion (FIG. 3d). Alternatively, the heteroconfigurational oligonucleotide may be labelled. FIG. 3a shows an embodiment in which the L-form portion comprises a label. FIG. 3b shows an embodiment in which the D-form portion comprises a label. FIG. 3c shows an embodiment in which a heteroconfigurational oligonucleotide is hybridized to a complementary polynucleotide that comprises several labels for detection. The RNA may then be hydrolyzed under hydrolysis conditions such as high pH, RNase cleavage, and/or certain salts such as Mg⁺² and Zn⁺². The resultant labelled cDNA is then purified to remove excess primers and nucleotides by a spin column method (Qiagen), silica gel treatment, ultrafiltration (Microcon), or precipitation.

In some embodiments, the present invention also includes a high-throughput assay for the analysis of many mRNA sequences. Gene specific reverse transcriptase primer can be designed and synthesized, which enable selective copying and amplification. Each specific sequence can be part of, or the entirety of, the D-form sequence portion of a heteroconfigurational oligonucleotide. Each gene specific sequence can be tagged with a specific L-form sequence portion in the heteroconfigurational oligonucleotide. The L-form complement to the specific L-form sequence portion in the heteroconfigurational oligonucleotide can be contained in an immobilized oligonucleotide. Where a limited number of mRNA sequences are to be detected, e.g. about 100, this number of immobilized oligonucleotides constitute an array that can be used for any sample. Arrays of L-form can be reused multiple times with appropriate denaturing wash routines, or they may be used once and discarded.

In arrays where D-form immobilized oligonucleotides that “capture” D-form nucleic acid analytes (e.g. cDNA) by sequence-specific hybridization, the problem of cross-hybridization may occur. False positive results can arise by detecting signal due to non-specific binding of D-form nucleic acid analytes to D-form immobilized oligonucleotides which are not complementary and contain one or more mismatches. In addition to false positives, a persistent and high-level of background signal may limit detectability, sensitivity, and otherwise obscure results. The present invention provides L-form sequences that do not effectively hybridize to D-form sequences, even those which are complementary in the Watson-Crick or Hoogsteen base-pairing sense. In other words, L-DNA does not effectively cross hybridize with D-DNA. Thus, the L-form binding motif provides orthogonality, i.e. another dimension of specificity in the molecular recognition properties of nucleic acids. Also, because L-form nucleic acids are not substrates for nuclease degradation, the universal array may have the additional advantage of greater stability, ruggedness, robustness, and storage life.

Kits

By configuring standard primer pairs and probes as reagent kits and robotic dispensing into the vessels (i.e. tubes, wells, array loci, or spots), high-throughput assays for profiling single-nucleotide polymorphisms (SNP), allelic discrimination, or disease related genes can be performed.

### EXAMPLES

The invention having been described, the following Examples are offered by way of illustration, and not limitation.

### Example 1

### Synthesis of Heteroconfigurational Oligonucleotides

L-DNA phosphoramidites were purchased from ChemGenes (Ashland Technology Centre, 200 Homer Avenue, Ashland, Mass. 01721). The L-DNA-D-DNA oligonucleotides were synthesized on an ABI 394 DNA/RNA synthesizer using a 0.2 mmol DNA cycle following the standard synthesis cycle (ABI3948, Nucleic Acid Synthesis and Purification System, Perkin Elmer Corp. 1995, Chapter 4: Automated Chemistry). The standard DNA amidites were placed at positions 1-4 and the L-DNA amidites at 5-8. After the synthesis the oligos were cleaved from the support with ammonium hydroxide and deprotected overnight at 55 degrees C. The ammonia was removed and the pellet dissolved in water. The concentration of the samples was determined by UV spectroscopy and stock solutions of 100 mM in ddH2O were prepared.

### Example 2

### L-DNA Binding on Array

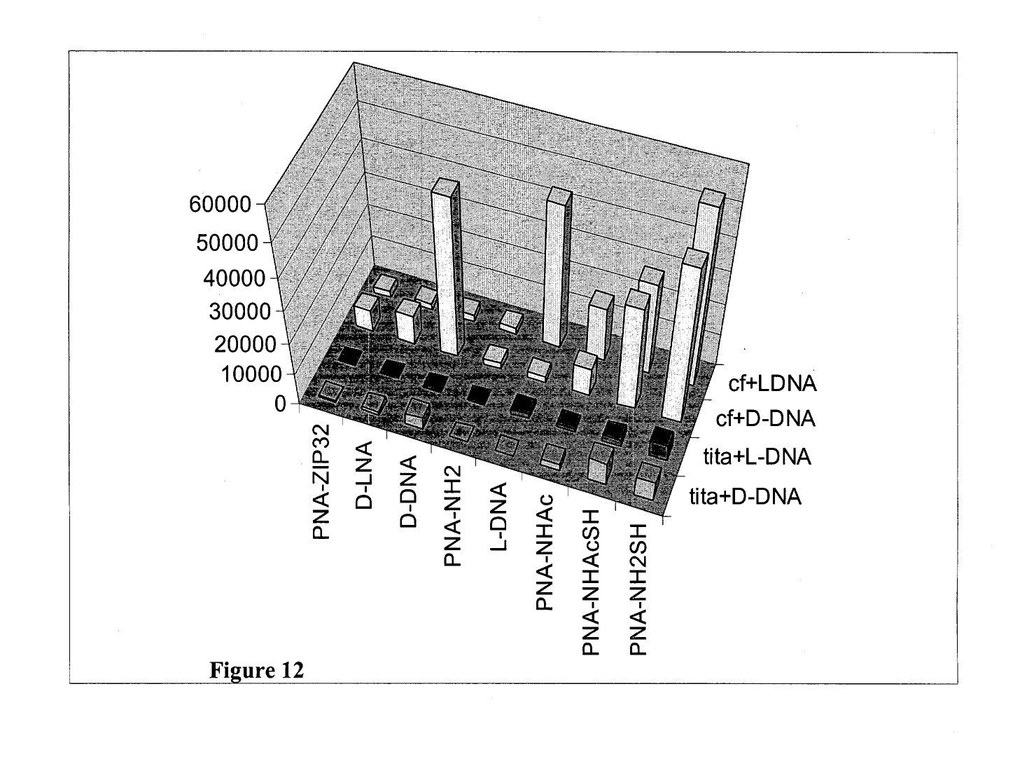

FIG. 12 shows results of experiments in which 8×6 arrays of 8 different probes (6 replicates each) were prepared (immobilized probes: PNA_ZIP32 (non-complementary control), D-LNA, D-DNA, PNA-NH₂, L-DNA, PNANHAc, PNANHAcSH, and PNANH₂SH), followed by hybridization with either of four different oligonucleotide solutions containing either oligo X-SM032 05b CF (L-DNA, “cf”), oligo X-SM032 04b CF (D-DNA, “cf”), oligo X-SM032 02b TF (L-DNA, “tita”), or oligo X-SM032 01 TF (D-DNA, “tita”). The first two probes contain sequences that are complementary to the sequences of the immobilized probes (if configuration is ignored). The second two probes contain sequences that are not complementary to any of the immobilized probes.

As can be seen from FIG. 12, the “cf” L-DNA probe hybridized to the complementary L-DNA and the last three PNA probes, but not to the other probes. The “cf” D-DNA probe hybridized to the complementary D-DNA and the last three PNA probes, but not to the other probes. Neither the D nor the L “tita” probes bound significantly to any of the immobilized probes, since there was no sequence complementarity.

All publications, patents and patent applications mentioned in this specification are herein incorporated by reference to the same extent as if each individual publication, patent or patent application was specifically and individually indicated to be incorporated by reference.

The invention now having been fully described, it will be apparent to one of ordinary skill in the art that many changes and modifications can be made thereto without departing from the spirit or scope of the invention.

1. A polynucleotide composition comprising
a heteroconfigurational polynucleotide comprising a D-form polynucleotide sequence portion and an L-form polynucleotide sequence portion that is covalently linked to the D-form polynucleotide sequence portion.

2. The composition of claim 1, wherein the L-form polynucleotide sequence portion comprises 5 to 50 L-nucleotides.

3. The composition of claim 1, wherein the D-form polynucleotide sequence portion comprises 5 to 50 D-nucleotides.

4. The composition of claim 3, wherein the L-form polynucleotide sequence portion comprises 5 to 50 L-nucleotides.

5. The composition of any one of the preceding claims, wherein the L-form polynucleotide sequence portion comprises at least one L-form 2′-4′ LNA nucleotide.

6. The composition of any one of claims 1 to 4, wherein the L-form polynucleotide sequence portion comprises at least one L-form nucleotide comprising a 1′-α-anomeric nucleotide or a 4′-α-anomeric nucleotide.

7. The composition of any one of claims 1 to 4, wherein the L-form polynucleotide sequence portion comprises at least one L-form nucleotide comprising ribose, arabinose, xylose, or pyranose, in the 1′-β anomeric configuration.

8. The composition of any one of claims 1 to 4, wherein the L-form polynucleotide sequence portion comprises at least one L-form nucleotide comprising ribose, arabinose, xylose, or pyranose, in the 1′-α anomeric configuration.

9. The composition of any one of claims 1 to 4, wherein the L-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose.

10. The composition of any one of the preceding claims, wherein the D-form polynucleotide sequence portion comprises at least one D-form 2′-4′ LNA nucleotide.

11. The composition of claim any one of claims 1 to 9, wherein the D-form polynucleotide sequence portion comprises at least one L-form nucleotide comprising a 1′-α-anomeric nucleotide or a 4′-α-anomeric nucleotide.

12. The composition of any one of claims 1 to 9, wherein the D-form polynucleotide sequence portion comprises at least one L-form nucleotide comprising ribose, arabinose, xylose, or pyranose, in the 1′-β anomeric configuration.

13. The composition of any one of claims 1 to 9, wherein the D-form polynucleotide sequence portion comprises at least one L-form nucleotide comprising ribose, arabinose, xylose, or pyranose, in the 1′-α anomeric configuration.

14. The composition of any one of the preceding claims, wherein the D-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose.

15. The composition of any one of the preceding claims, wherein at least one of the D-form polynucleotide sequence portion and the L-form polynucleotide sequence portion comprises an internucleotide linkage selected from a 2-aminoethylglycine, a phosphorothioate, a phosphorodithioate, a phosphotriester, and a phosphoramidate.

16. The composition of any one of the preceding claims, wherein the heteroconfigurational polynucleotide comprises a nucleobase selected from uracil, thymine, cytosine, adenine, 7-deazaadenine, guanine, and 7-deazaguanosine.

17. The composition of claim of any one of claims 1 to 15, wherein the heteroconfigurational polynucleotide comprises a nucleobase selected from 2,6-diaminopurine, hypoxanthine, pseudouridine, C-5-propyne, isocytosine, isoguanine, and 2-thiopyrimidine.

18. The composition of claim any one of the preceding claims, which comprises a first complementary polynucleotide that is hybridized to the L-form polynucleotide sequence portion.

19. The composition of claim 18, wherein the first complementary polynucleotide comprises at least one L-form nucleotide.

20. The composition of claim 18, wherein the first complementary polynucleotide comprises at least one L-form 2′ deoxyribose or 2′-4′ LNA nucleotide.

21. The composition of claim 18, wherein the first complementary polynucleotide comprises at least two peptide nucleic acid subunits.

22. The composition of any one of claims 18 to 21, wherein the first complementary polynucleotide is attached to a solid support.

23. The composition of claim 22, wherein the solid support comprises polystyrene, glass, silica gel, silica, polyacrylamide, polyacrylate, hydroxyethyl-methacrylate, polyamide, polyethylene, polyethyleneoxy, or nylon.

24. The composition of claim 22 or claim 23, wherein the solid support comprises a small particle, a bead, a membrane, a frit, a slide, a plate, a micromachined chip, an alkanethiol-gold layer, a non-porous surface, an addressable array, or a gel.

25. The composition of claim 24, wherein the solid support comprises a bead.

26. The composition of claim 25, wherein the solid support comprises a polystyrene bead.

27. The composition of claim 23, wherein the solid support comprises a nylon membrane.

28. The composition of claim 24, wherein the solid support comprises a small particle selected from a nanoparticle, a microsphere, or a liposome.

29. The composition of claim 22 wherein the solid support comprises glass.

30. The composition of any one of claims 22 to 29, wherein the first complementary polynucleotide is attached to the support via a cleavable linker.

31. The composition of claim 30, wherein the cleavable linker comprises a carbonyl group through which the first complementary polynucleotide is linked to the support.

32. The composition of any one of the preceding claims, which comprises a second complementary polynucleotide that is hybridized to the D-form polynucleotide sequence portion.

33. The composition of any one of the preceding claims, which comprises a detectable label.

34. The composition of claim 33, wherein the label comprises a fluorescent dye, a fluorescence quencher, an energy-transfer pair, a quantum dot, or a chemiluminescent precursor.

35. The composition of claim 34, wherein the label comprises a fluorescein, a rhodamine, or a cyanine.

36. The composition of any one of claims 33 to 35, wherein the label is attached to a second complementary polynucleotide that is hybridized to the D-form polynucleotide sequence portion.

37. An array of different-sequence polynucleotides comprising 5 to 100 L-nucleotides, wherein the polynucleotides are immobilized at addressable locations on a solid support.

38. The array of claim 37 wherein the solid support comprises polystyrene, glass, silica gel, silica, polyacrylamide, polyacrylate, hydroxyethylmethacrylate, polyamide, polyethylene, polyethyleneoxy, or nylon.

39. The array of claim 37 wherein the solid support comprises a small particle, a bead, a membrane, a frit, a slide, a plate, a micromachined chip, an alkanethiol-gold layer, a non-porous surface, an addressable array, or a gel.

40. The array of claim 39, wherein the solid support comprises a bead.

41. The array of claim 40, wherein the solid support comprises a polystyrene bead.

42. The array of claim 39, wherein the solid support comprises a nylon membrane.

43. The array of claim 39, wherein the solid support comprises a small particle selected from a nanoparticle, a microsphere, or a liposome.

44. The array of claim 39, wherein solid support comprises glass.

45. The array one of claims 37 to 44, wherein the first complementary polynucleotide is attached to the support via a cleavable linker.

46. The array of claim 45, wherein the cleavable linker comprises a carbonyl group through which the first complementary polynucleotide is linked to the support.

47. The array of any one of claims 37 to 46, wherein the solid support is configured as a 96 well format.

48. The array of any one of claims 37 to 47, wherein at least one polynucleotide comprises a label.

49. The array of claim 48, wherein the label comprises a fluorescent dye, a quencher, an energy-transfer dye, a quantum dot, digoxigenin, biotin, a mobility-modifier, a polypeptide, a hybridization-stabilizing moiety, or a chemiluminescent precursor.

50. The array of claim 49 wherein at least one immobilized polynucleotide comprises the structure: 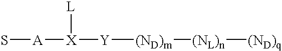
wherein S is a solid support;
A is a linker;
X is a linker with three or more attachment sites;
Y is O, NH, NR, or S, where R is selected from C₁-C₆ alkyl, C₁-C₆ substituted alkyl, C₅-C₁₄ aryl, and C₅-C₁₄ substituted aryl;
L is hydrogen or a label;
N_{L} is a sequence of L-form nucleotides;
N_{D} is a sequence of D-form nucleotides;
m is an integer from 0 to 100; and
n is an integer from 5 to 100; and
q is an integer from 0 to 100.

51. The array of claim 50, wherein A is a cleavable linker.

52. The array of claim 51, wherein A comprises one or more of the structures:

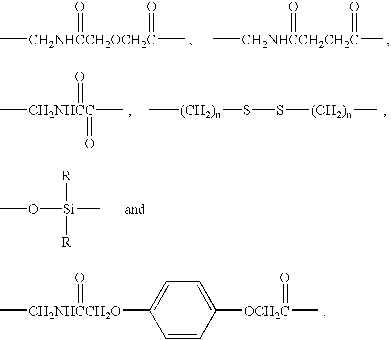

53. The array of claim 50, wherein (N_{D})ₘ and (N_{L})ₙ, and (N_{L})ₙ and (N_{D})_{q}, are linked to each other by linkers.

54. The array of claim 53, wherein the linker comprises one or more ethyleneoxy units.

55. The array of claim 50, wherein m=0.

56. The array of claim 50, wherein m=q=0.

57. A method of forming a polynucleotide hybrid comprising
providing a heteroconfigurational polynucleotide comprising a D-form polynucleotide sequence portion and an L-form polynucleotide sequence portion that is covalently linked to the D-form polynucleotide sequence portion, and hybridizing the heteroconfigurational polynucleotide to a first complementary polynucleotide to form a duplex between the first complementary polynucleotide and the L-form polynucleotide sequence portion.

58. The method of claim 57, wherein the L-form polynucleotide sequence portion comprises 5 to 50 L-nucleotides.

59. The method of claim 57, wherein the D-form polynucleotide sequence portion comprises 5 to 50 D-nucleotides.

60. The method of claim 59, wherein the L-form polynucleotide sequence portion comprises 5 to 50 L-nucleotides.

61. The method of any one of claims 57 to 60, wherein the L-form polynucleotide sequence portion comprises at least one L-form 2′-4′ LNA nucleotide.

62. The method of any one of claims 57 to 60, wherein the L-form polynucleotide sequence portion comprises at least one L-form nucleotide comprising a 1′-α-anomeric nucleotide or a 4′-α-anomeric nucleotide.

63. The method of any one of claims 57 to 60, wherein the L-form polynucleotide sequence portion comprises at least one L-form nucleotide comprising ribose, arabinose, xylose, or pyranose, in the 1′-β anomeric configuration.

64. The method of any one of claims 57 to 60, wherein the L-form polynucleotide sequence portion comprises at least one L-form nucleotide comprising ribose, arabinose, xylose, or pyranose, in the 1′-α anomeric configuration.

65. The method of any one of claims 57 to 60, wherein the L-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose.

66. The method of any one of claims 57 to 65, wherein the D-form polynucleotide sequence portion comprises at least one D-form 2′-4′ LNA nucleotide.

67. The method of claim any one of claims 57 to 65, wherein the D-form polynucleotide sequence portion comprises at least one L-form nucleotide comprising a 1′-α-anomeric nucleotide or a 4′-α-anomeric nucleotide.

68. The method of any one of claims 57 to 65, wherein the D-form polynucleotide sequence portion comprises at least one L-form nucleotide comprising ribose, arabinose, xylose, or pyranose, in the 1′-β anomeric configuration.

69. The method of any one of claims 57 to 65, wherein the D-form polynucleotide sequence portion comprises at least one L-form nucleotide comprising ribose, arabinose, xylose, or pyranose, in the 1′-α anomeric configuration.

70. The method of any one of claims 57 to 69, wherein the D-form polynucleotide sequence portion comprises at least one L-form nucleotide comprising ribose, 2′-deoxyribose, 2′,3′-dideoxyribose, 2′-fluororibose, 2′-chlororibose, or 2′-O-methylribose.

71. The method of any one of claims 57 to 70, wherein at least one of the D-form polynucleotide sequence portion and the L-form polynucleotide sequence portion comprises an internucleotide linkage selected from a 2-aminoethylglycine, a phosphorothioate, a phosphorodithioate, a phosphotriester, and a phosphoramidate.

72. The method of any one of claims 57 to 72, wherein the first complementary polynucleotide comprises at least one L-form nucleotide.

73. The method of any one of claims 57 to 72, wherein the first complementary polynucleotide comprises at least one L-form 2′ deoxyribose or 2′-4′ LNA nucleotide.

74. The method of any one of claims 57 to 72, wherein the first complementary polynucleotide comprises at least two peptide nucleic acid subunits.

75. The method of any one of claims 57 to 72, wherein unhybridized first complementary polynucleotide is separated from said hybrid.

76. The method of claim 75 further comprising detecting the hybrid.

77. The method of any one of claim 57 to 76, which comprises primer extension of the heteroconfigurational polynucleotide.

78. The method of any one of claim 57 to 76, which comprises cleavage of the heteroconfigurational polynucleotide by a nuclease enzyme.

79. The method of any one of claim 57 to 76, which comprises ligation of a heteroconfigurational polynucleotide to a polynucleotide that is hybridized adjacent to an end of the heteroconfigurational polynucleotide.

80. The method of any one of claims 57 to 79, wherein the hybrid is immobilized on a solid support.

81. A kit comprising
a heteroconfigurational polynucleotide in accordance with any one of claims 1 to 17, and
a solid support to which is attached at least one polynucleotide comprising an L-form polynucleotide sequence portion that is complementary to the L-form polynucleotide sequence portion in the heteroconfigurational polynucleotide.

82. The kit of claim 81, comprising a plurality of solid supports, each support being attached to a heteroconfigurational polynucleotide comprising an L-form polynucleotide sequence portion comprising a unique sequence that is distinct from the sequences of the L-form polynucleotide sequence portions in the other solid supports of said plurality.

83. The kit of claim 81, which comprises an addressable array of heteroconfigurational polynucleotide at different locations, each polynucleotide comprising an L-form heteroconfigurational polynucleotide sequence portion comprising a unique sequence that is distinct from the sequences of the L-form polynucleotide sequence portions in the heteroconfigurational polynucleotides at other locations on the array.

84. The kit of any one of claims 81 to 83, wherein the kit comprises at least 10 different heteroconfigurational polynucleotides each comprising a unique sequence that is distinct from the L-form polynucleotide sequence portions in the other heteroconfigurational polynucleotides.

85. The kit of any one of claims 84, wherein the kit comprises at least 100 different heteroconfigurational polynucleotides each comprising a unique sequence that is distinct from the L-form polynucleotide sequence portions in the other heteroconfigurational polynucleotides.
